(19) United States
(12) Patent Application Publication
(10) Pub. No.: US 2022/0251627 A1
(43) Pub. Date: Aug. 11, 2022

(54) COMPOSITIONS FOR THE STABILIZATION OF CELL-FREE NUCLEIC ACIDS AND METHODS THEREOF

(71) Applicant: ZYMO RESEARCH CORPORATION, Irvine, CA (US)

(72) Inventors: Hanjun KIM, Irvine, CA (US); Ryan KEMP, Irvine, CA (US); Xi-Yu JIA, Newport Beach, CA (US)

(73) Assignee: ZYMO RESEARCH CORPORATION, Irvine, CA (US)

(21) Appl. No.: 17/621,958

(22) PCT Filed: Jun. 26, 2020
(86) PCT No.: PCT/US2020/039849

§ 371 Date: Dec. 22, 2021

Related U.S. Application Data
(60) Provisional application No. 62/868,795, filed on Jun. 28, 2019.

(51) Int. Cl. C12Q 1/6806; G01N 1/40

(52) U.S. Cl. CPC C12Q 1/6806; G01N 1/40

(57) ABSTRACT

Provided herein are aqueous compositions, kits and methods for the stabilization of cell-free nucleic acids. Provided herein are aqueous compositions for the stabilization of a cell-free nucleic acid (cfNA) population in a blood sample. In certain embodiments, the aqueous composition comprises a polyvinylpyrrolidone (PVP), an apoptosis inhibitor and one or more eryptosis inhibitors.

### CROSS REFERENCE TO RELATED APPLICATIONS

This application claims the benefit of U.S. Provisional Application No. 62/868,795, filed Jun. 28, 2019, which is incorporated by reference herein in its entirety.

### SEQUENCE LISTING

This application incorporates by reference in its entirety a Sequence Listing submitted with this application in ASCII text format, entitled “14589-001-228_SEQ_LISTING.txt,” created on Jun. 21, 2020, and is 1,565 bytes in size.

### 1. FIELD

This application relates generally to aqueous compositions for the stabilization of cell-free nucleic acids. Kits and methods of making and using the aqueous compositions are also provided herein.

### 2. BACKGROUND

Cell-free nucleic acids (cfNA), including cell-free DNA (cfDNA) and cell-free RNA (cfRNA), are recognized as important biomarkers for monitoring patient health. For example, cfNA can be a useful tool for the detection and early diagnosis of diseases, such as cancer. In addition, analysis of cfNA can provide a non-invasive method of monitoring the health of mothers and fetuses during pregnancy.

Approximately half of total blood volume comprises cells of various types, including various white blood cells (WBCs) and red blood cells (RBCs). Whole blood deteriorates quickly ex vivo, resulting in cell lysis and release of cellular nucleic acids. cfRNA. Both cfDNA and cfRNA are present in minute amounts in the extracellular environment, or plasma fraction, of blood and must be protected from contamination by cellular DNA and RNA. There is a need for methods of stabilizing the highly dilute amounts of cell-free NA (cfNA) circulating in plasma from becoming contaminated by lysis and spillage of cellular NA during storage of blood samples.

### 3. SUMMARY

Provided herein are aqueous compositions for the stabilization of a cell-free nucleic acid (cfNA) population in a blood sample. In certain embodiments, the aqueous composition comprises a polyvinylpyrrolidone (PVP), an apoptosis inhibitor and one or more eryptosis inhibitors. In certain embodiments, the PVP has a weight average molecular weight of from about 10,000 to about 40,000 Daltons, as determined by an experimental technique, for example, a light scattering technique. In certain embodiments, the PVP is present in an amount of about 5% w/v to about 30% w/v of the aqueous composition.

In certain embodiments, the apoptosis inhibitor is a caspase inhibitor. In certain embodiments, the apoptosis inhibitor is selected from the group consisting of quinolyl-valyl-O-methylaspartyl-[-2,6-difluorophenoxy]-methyl ketone (Q-VD-OPh), carbobenzoxy-valyl-alanyl-aspartyl-[O-methyl]-fluoromethylketone (Z-VAD-FMK), and Boc-Asp(OMe)-fluoromethyl ketone (BOC-D-FMK). In certain embodiments, the apoptosis inhibitor is quinolyl-valyl-O-methylaspartyl-[-2,6-difluorophenoxy]-methyl ketone (Q-VD-OPh). In certain embodiments the Q-VD-OPh has a concentration of about 50 μM to about 5000 μM.

In certain embodiments, the one or more eryptosis inhibitors are selected from the group consisting of an inhibitor of increased intracellular Ca2+ activity, an inhibitor of ceramide formation, and an inhibitor of ATP depletion. In certain embodiments, the one or more eryptosis inhibitors are selected from the group consisting of adenosine, amitriptyline, caffeine, a catecholamine, D4476, dibutyryl-cGMP, dithiothreitol, ethylisopropylamiloride, erythropoietin, flufenamic acid, furosemide, glutathione, 7-monohydroxyethylrutoside, N-acetylcysteine, naringin, 1,2,3,4-tetrahydro-6-nitro-2,3-dioxo-benzo[f]quinoxaline-7-sulfonamide, 6-cyano-7-nitroquinoxaline-2,3-dione (CNQX), niflumic acid, nitroprusside, 5-nitro-2-(3-phenylpropylamino)benzoic acid (NPPB), papanonoate, P38 Inh III, resveratrol, (R)-DRF503, salidroside, 4-(4-fluorophenyl)-2-(4-methylsulfinylphenyl)-5-(4-pyridyl)imidazole (SB203580), Staurosporine, Trolox, urea, vitamin E, xanthohumol, and Zidovudine. In certain embodiments, the one or more eryptosis inhibitors are selected from the group consisting of urea, naringin and caffeine. In certain embodiments, where one or more of urea, naringin and caffeine are present in the aqueous composition, the urea has a concentration of about 4 mM to about 650 mM, the naringin has a concentration of about 40 nM to about 40 μM, and/or the caffeine has a concentration of about 2 μM to about 500 μM.

In certain embodiments the aqueous composition disclosed herein further comprises polyethylene glycol dimethyl ether (dmPEG). In certain embodiments, the dmPEG has a weight average molecular weight of from about 250 to about 4000 Daltons, as determined by an experimental technique, for example, a light scattering technique. In certain embodiments, the dmPEG is present in an amount about 5% w/v to about 30% w/v of the aqueous composition.

In certain embodiments, the aqueous composition disclosed herein is a saline composition.

In certain embodiments, the aqueous composition further comprises an anticoagulant. In certain embodiments, the anticoagulant is a chelator. In certain embodiments, the chelator is EDTA dipotassium salt (K₂EDTA). In certain embodiments, the chelator is EDTA dipotassium salt (K₂EDTA). In certain embodiments, the K₂EDTA has a concentration of about 10 mM to about 1000 mM.

In certain embodiments, the aqueous composition disclosed herein further comprises a sugar. In certain embodiments, the sugar is selected from the group consisting of glucose, lactose, fructose, and galactose. In certain embodiments, the sugar is glucose. In certain embodiments, the concentration of the glucose is about 10 mM and about 1000 mM.

In certain embodiments, the aqueous compositions disclosed herein are used for research or diagnostic purposes.

Also provided herein is a kit for stabilizing a cell-free nucleic acid (cfNA) population in a blood sample comprising an aqueous composition disclosed herein, and further comprising a blood collection container and instructional materials.

### 4. BRIEF DESCRIPTION OF THE FIGURES

FIG. 1A. Shown is a microcapillary gel image of cfDNA isolated from plasma from blood samples treated with caspase inhibitor.

FIG. 1B. Shown is an agarose gel image of genomic DNA isolated from blood pellet from blood samples treated with caspase inhibitor.

FIG. 2A. Shown is a hemolysis assay consisting of hemolysis peak absorbance values of plasma from blood samples treated with polyethylene glycol, caspase inhibitor, or combinations of the two.

FIG. 2B. Shown is a microcapillary gel image of cfDNA isolated from plasma from blood samples treated with polyethylene glycol, caspase inhibitor, or combination of the two.

FIG. 2C. Shown is a genomic DNA isolated from blood pellet isolated by centrifugation from blood samples treated with polyethylene glycol, caspase inhibitor, or combinations of the two.

FIG. 3A. Shown is a hemolysis assay consisting of hemolysis peak absorbance values of plasma from blood samples treated with various polymer types.

FIG. 3B. Shown is a microcapillary gel image of cfDNA isolated from plasma from blood samples treated with various polymer types.

FIG. 3C. Show is a microcapillary gel image of cfDNA isolated from plasma from blood samples treated with various polymer types.

FIG. 3D. Show is a microcapillary gel image of cfDNA isolated from plasma from blood samples treated with various polymer types.

FIG. 3E. Shown is a hemolysis assay consisting of hemolysis peak absorbance values of plasma from blood samples treated with titrated concentrations of several polymers.

FIG. 4A. Shown are average cycle threshold values of a cfDNA fragment, Sat2, from plasma from blood samples treated with preservative containing polyvinylpyrrolidone polymer for extended period of time.

FIG. 4B. Shown is a hemolysis assay consisting of hemolysis peak absorbance values of plasma from blood samples treated with preservative containing polyvinylpyrrolidone polymer for extended period of time.

FIG. 4C. Shown is an agarose gel image of genomic DNA isolated from blood pellets from blood samples treated with preservative containing polyvinylpyrrolidone polymer for extended period of time.

FIG. 5A. Shown are average cycle threshold values of a cfDNA fragment, Sat2, from plasma from blood samples treated with preservative containing polyvinylpyrrolidone polymer and with preservatives from different vendors.

FIG. 5B. Shown are fold changes from FIG. 5A average cycle threshold values of a cfDNA fragment, Sat2, from plasma from blood samples treated with preservative containing polyvinylpyrrolidone polymer and with preservatives from different vendors.

FIG. 6A. Shown is a hemolysis assay consisting of hemolysis peak absorbance values of plasma from blood samples treated with preservative containing different polymer concentrations and combinations.

FIG. 6B. Shown are average cycle threshold values of a cell-free microRNA, hsa-miR-16-5p, from plasma from blood samples treated with preservative containing different polymer concentrations and combinations.

FIG. 6C. Shown are average cycle threshold values of a cell-free microRNA enriched in red blood cell, hsa-miR-451a, from plasma from blood samples treated with preservative containing different polymer concentrations and combinations.

FIG. 7A. Shown is a hemolysis assay consisting of hemolysis peak absorbance values of plasma from blood samples treated with preservative with addition of various eryptosis inhibitors.

FIG. 7B. Shown are average cycle threshold values of red blood cell enriched microRNA, hsa-miR-451a, from cfRNA isolated from blood samples treated with preservative with addition of various eryptosis inhibitors.

FIG. 7C. Shown are average cycle threshold values of red blood cell enriched microRNA, hsa-miR-451a, from cfRNA isolated from blood samples treated with preservative with addition of various eryptosis inhibitors.

### 4.1 DEFINITIONS

As used herein, the terms “nucleic acid” or “nucleic acids” refer to a polymer composed of nucleotide units, including deoxyribonucleic acid (“DNA”) and ribonucleic acid (“RNA”), typically found in nature in chromosomes, chromatin, mitochondria, cytoplasm, ribosomes, bacteria, fungi, viruses or extracellularly (e.g., cell-free nucleic acids). Non-limiting examples of nucleic acids include chromatin, miRNA, cDNA, DNA, single stranded DNA, double stranded DNA, genomic DNA (gDNA), plasmid DNA, or RNA. In certain embodiments, nucleic acid can be double stranded or single stranded. In certain embodiments, a nucleic acid can be intracellular. In certain embodiments, a nucleic acid can be extracellular (e.g., cell-free). In certain embodiments, a nucleic acid can be fragmented. The methods and compositions disclosed herein are generally useful for stabilizing and/or protecting nucleic acids (e.g., circulating and/or cell-free nucleic acids).

As used herein, the term “cell-free nucleic acid (cfNA)” refers to circulating, extracellular or free-floating nucleic acid. In certain embodiments, a cfNA population includes nucleic acids existing outside of any intact or partially intact cell. In certain embodiments, cfNA includes nucleic acids existing outside of any intact or partially intact cell, but within a cellular or cell-like component, e.g., within a membrane structure, a mitochondria-like structure, a lipid membrane vesicle, etc. In certain embodiments, cfNA can be bound to one or more proteins (e.g., histones).

As used herein, the term “cell-free ribonucleic acid (cfRNA)” is used interchangeably with circulating RNA or extracellular RNA and refers to RNA (e.g. mRNA) present within the cell-free fraction of a sample. The cell-free RNA described herein is not typically found in intact cells (e.g., found in uncompromised plasma membrane) but may be associated with particles (e.g. placenta-derived syncytiotrophoblast microparticles, see Rusterholz C. et al., Fetal Diagn Ther. (2007) 22(4):313-7; or apoptotic bodies, see Hasselmann D. O. et al., Clin Chem. (2001) 47:1488-1489). In certain embodiments the cell-free RNA is intact (e.g. not fragmented).

As used herein, the term “cell-free deoxyribonucleic acid (cfDNA)” is used interchangeably with circulating DNA or extracellular DNA and often refers to DNA that has been extruded from cells, or DNA that has been released from necrotic and/or apoptotic cells. In certain embodiments, the cfDNA can be any cfDNa known in the art. In certain embodiments, the cfDNA can be maternal nucleic acids and/or fetal nucleic acids (e.g., nucleic acids of a pregnant female subject and the nucleic acids of the fetus being carried by the pregnant female, respectively). In certain embodiments, the cfDNA can be circulating DNA released from diseased cells (e.g., cancer cells).

As used herein, a “subject” can be a mammal such as a non-primate (e.g., cows, pigs, horses, cats, dogs, rats, etc.) or a primate (e.g., monkey and human). In specific embodiments, the subject is a human. In certain embodiments, the subject is a mammal (e.g., a human) having or potentially having a disease, disorder or condition described herein. In certain embodiments, the subject is a mammal (e.g., a human) at risk of developing a disease, disorder or condition described herein.

As used herein, the term “aqueous composition” is a composition in an aqueous solution (e.g., a saline solution). Thus, as used herein, in some embodiments, an “aqueous composition” is a “saline composition,” wherein a “saline composition” is a composition in a saline solution. An “aqueous composition” may be a homogenous mixture of only one phase, but it is also within the scope of the present disclosure that a solution comprises solid components such as e.g. precipitates.

As used herein, the term “cell-free nucleic acid population” (cfNA population), as used herein, refers to a collective of one or more different cell-free nucleic acids. A biological sample, for example, a cell-containing biological sample (e.g., blood) can comprise a characteristic (e.g., unique) cfNA population. Thus, one of skill in the art would consider that the type, kind and/or the amount of one or more cfNAs comprised in the cfNA population of a specific sample are important sample characteristics. As used herein, the term “cfNA population” can comprise one or more forms of cell-free nucleic acid (e.g., double-stranded DNA, single-stranded DNA and single-stranded RNA).

As used herein, the term “molecular weight” generally refers to the mass or average mass of a material, for example, the weight average molecular weight of a material. Molecular weight can be calculated from the formula of a compound. If the compound is a polymer or oligomer, the molecular weight can refer to the relative average chain length or relative chain mass of the bulk polymer, as determined by an experimental technique. In practice, the molecular weight of polymers and oligomers can be estimated or characterized in various ways including gel permeation chromatography (GPC), membrane osmometry, viscosity analysis, NMR analysis or mass spectrometry.

In certain embodiments, the weight average molecular weight of a polymer (e.g., PVP) disclosed herein can be determined using any methods known in the art. As a non-limiting example, in certain embodiments, the polymer provided herein can have a weight average molecular weight determined by light scattering methods. Light scattering methods for polymer characterization are known in the art (see, e.g., Zimm B. H. et al., Journal of Chemical Physics, (1948) 16(12):1099-1116). Without being bound by theory, when paired with size exclusion chromatography and a concentration detection method such as differential refractive index, light scattering detection can enable precise characterization of the molecular weight distribution of polydisperse polymer samples. See, for example, Wyatt P. J. et al., Analytica Chimica Acta, (1993) 272:1-40.

It is understood that polymer nomenclature usually applies to idealised representations; minor structural irregularities are ignored. Weight average molecular weights of polymers are approximations. As used herein and understood in the art, a number following the name of a polymer refers to the weight average molecular weight. For example, a PVP polymer of about 10,000 MW can be depicted as PVP-10000.

As used herein, the term “stabilization” refers to the preservation, protection or maintenance of a biomolecule (e.g., cfNA) or composition comprising a biomolecule (e.g., blood and/or blood mixed with an aqueous composition disclosed herein), wherein the fragmentation, degradation and/or modification of one or a plurality of cfNAs or cfNA populations that are present in the biological sample is substantially prevented. It is understood that compositions and methods that are effective to stabilize one class of biomolecules may or may not be effective in stabilizing others. In certain embodiments, target nucleic acid (e.g., cfNA) can be stabilized such that it will be of sufficient quality and quantity for subsequent processes such as amplification, hybridization or sequencing, following storage and/or incubation (e.g., at ambient temperature) for a desired amount of time.

As used herein, the term “apoptosis” refers to a regulated cascade of biochemical events which lead to cell death or suicide program which can be characterized by a condensation and subsequent fragmentation of the cell nucleus. In response to a triggering stimulus, cells undergo a cascade of events including cell shrinkage, blebbing of cell membranes and chromatic condensation and fragmentation. These events can culminate in cell conversion to clusters of membrane-bound particles (apoptotic bodies), which can thereafter be engulfed by macrophages. Hallmarks of apoptosis include, but are not limited to, morphological changes, cell shrinkage, nuclear and cytoplasmic condensation, and alterations in plasma membrane topology. Biochemically, apoptotic cells can be characterized by increased intracellular calcium concentration, fragmentation of chromosomal DNA, and expression of novel cell surface components.

As used herein, the term “apoptosis inhibitor” refers to an agent or compound (e.g., a chemical or biological agent) that can inhibit, reduce and/or prevent apoptotic processes in cells resulting in cell death by a programmed sequence of events. For example, the apoptosis inhibitor can make the cells more resistant to apoptotic stimuli. For example, in certain embodiments, an apoptosis inhibitor is useful to inhibit, reduce, or slow down, decrease, or stop the amount of apoptotic cell death, as measured using methods known to those of ordinary skill in the art, by, for example, 10%, 20%, 30%, 40%, 50%, 60%, 70%, 80%, 90%, 95%, or 100%, when compared to cells that are not subjected to the methods, compositions, and combinations of the present application. Apoptosis has been detected by a variety of accepted methods known in the art. As a non-limiting example, apoptosis can be detected using morphology, DNA fragmentation, enzymatic activity, and/or polypeptide degradation. In some morphological assays, methods can exploit nuclear chromatin condensation and the fragmentation of nuclear structures into apoptotic bodies. In some assays, inhibition of apoptosis can be measured by detection of DNA fragmentation that is characteristic of apoptosis or the release of fragmented nucleic acids from apoptotic cells. For example, apoptosis can be detected by the appearance of a DNA “ladder,” which is produced by endonuclease cleavage of chromosomal DNA into nucleosomal fragments (see, e.g., Compton M. M. et al., Cancer Metast. Rev. (1992) 11:105-119). It is understood that intracellular RNA, especially mRNA, can also be degraded during apoptosis.

In certain embodiments, the apoptosis inhibitor can be a caspase inhibitor, e.g., an inhibitor of one or more caspase enzymes (e.g., Q-VD-OPh ((3S)-5-(2,6-difluorophenoxy)-3-[[(2S)-3-methyl-2-(quinoline-2-carbonylamino)butanoyl]amino]-4-oxopentanoic acid), Z-VAD-FMK (methyl (3S)-5-fluoro-3-[[(2S)-2-[[(2S)-3-methyl-2-(phenylmethoxycarbonylamino)butanoyl]amino]propanoyl]amino]-4-oxopentanoate), Q-VD(OMe)-OPh ((S)-methyl 5-(2,6-difluorophenoxy)-3-((S)-3-methyl-2-(quinoline-2-carboxamido)butanamido)-4-oxopentanoate), or Boc-D-fmk (methyl 5-fluoro-3-[(2-methylpropan-2-yl)oxycarbonylamino]-4-oxopentanoate) or any derivatives thereof.

As used herein, the term “eryptosis” generally refers to a form of programmed cell death of erythrocytes, also known as “apoptosis of erythrocytes” or “suicidal erythrocyte death.” Erythrocytes do not undergo apoptosis, as they lack nuclei and mitochondria, key organelles involved in apoptosis. Eryptosis was first described in 2001 (Bratosin D. et al., Cell Death Differ (2001) 8:1143-1156) and the term eryptosis was proposed in 2005 (Lang K. et al. CPB (2005) 15:195-202). Eryptosis shares numerous similarities with apoptosis, including the purpose of destruction of damaged cells (Bratosin D. et al., Cell Death Differ. (2001) 8:1143-1156; Lang K. et al., CPB (2005) 15:195-202). As a non-limiting example, eryptosis can be characterized by increased intracellular calcium concentrations, phosphatidylserine translocation to the cell surface, cell shrinkage, and the scrambling, ruffling and blebbing of the cell membrane (Lau I. P. et al., Nanotoxicology (2012) 6:847-856; Maellaro E. et al., Acta Diabetol (2013) 50:489-495; Lang E. and Lang F. Semin Cell Dev Biol (2015) 39: 35-42; Bratosin D. et al., Cell Death Differ. (2001) 8:1143-1156; Lang K. et al., CPB 2005; 15, 195-202; Lang P A et al., Am J Physiol Cell Physiol (2003) 285:C1553-C1560).

As used herein, the term “eryptosis inhibitor” refers to an agent (e.g., a chemical or biological agent) that can inhibit or reduce eryptosis. For example, in certain embodiments, an eryptosis inhibitor is useful to inhibit, reduce, or slow down, decrease, or stop the amount of eryptotic cell death, as measured using methods known to those of ordinary skill in the art, by, for example, 10%, 20%, 30%, 40%, 50%, 60%, 70%, 80%, 90%, 95%, or 100%, when compared to cells that are not subjected to the methods, compositions, and combinations of the present application. An inhibitor of eryptosis can be any agent that can reduce or inhibit one or more characteristics of eryptosis described herein or known in the art. For example, in certain embodiments, an inhibitor of eryptosis can inhibit an increase in intracellular calcium concentrations (e.g., reduce intracellular calcium concentrations), phosphatidylserine translocation to the cell surface, cell shrinkage, and the scrambling, ruffling and blebbing of the cell membrane

As a non-limiting example, an eryptosis inhibitor may be any agents or derivatives thereof described herein or known in the art (e.g., as described in Lang and Lang, BioMed Research Int., 2015:513518). In certain embodiments, one or more eryptosis inhibitors may disclosed in Lang and Lang or known in the art can be used in embodiments of this invention. A non-limiting list of candidate eryptosis inhibitors includes: adenosine, amitriptyline, caffeine, a catecholamine, D4476, dibutyryl-cGMP, dithiothreitol, ethylisopropylamiloride, erythropoietin, flufenamic acid, furosemide, glutathione, 7-monohydroxyethylrutoside, N-acetylcysteine, naringin, 1,2,3,4-tetrahydro-6-nitro-2,3-dioxo-benzo[f]quinoxaline-7-sulfonamide, 6-cyano-7-nitroquinoxaline-2,3-dione (CNQX), niflumic acid, nitroprusside, 5-nitro-2-(3-phenylpropylamino)benzoic acid (NPPB), papanonoate, P38 Inh III, resveratrol, (R)-DRF503, salidroside, 4-(4-fluorophenyl)-2-(4-methylsulfinylphenyl)-5-(4-pyridyl)imidazole (SB203580), Staurosporine, Trolox, urea, vitamin E, xanthohumol, and Zidovudine.

### 4.2 EMBODIMENTS

Provided herein are aqueous compositions for the stabilization of a cell-free nucleic acid (cfNA) population in a blood sample, wherein the aqueous compositions comprise a polyvinylpyrrolidone (PVP), an apoptosis inhibitor and one or more eryptosis inhibitors. Further provided herein are kits comprising an aqueous compositions disclosed herein.

### 4.2.1 Polyvinylpyrrolidone

In certain embodiments, the aqueous compositions disclosed herein comprise a polyvinylpyrrolidone (PVP). PVP, also known as 1-ethenylpyrrolidin-2-one, povidone or polyvidone, is a synthetic polymer that consists essentially of linear chains include repeating units of N-vinyl-2-pyrrolidone, the degree of polymerization of which results in polymers of various molecular weights according to a Gaussian distribution.

In certain embodiments, the weight average molecular weight of the PVP provided herein can be from about 2,000 Daltons to about 200,000 Daltons (g/mol). In certain embodiments, the PVP has a weight average molecular weight of about 2,000 Daltons to about 4,000 Daltons, about 4,000 Daltons to about 6,000 Daltons, about 6,000 Daltons to about 8,000 Daltons, about 8,000 Daltons to about 10,000 Daltons, about 10,000 Daltons to about 12,000 Daltons, about 12,000 Daltons to about 14,000 Daltons, about 14,000 Daltons to about 16,000 Daltons, about 16,000 Daltons to about 18,000 Daltons, about 18,000 Daltons to about 20,000 Daltons, about 20,000 Daltons to about 30,000 Daltons, about 30,000 Daltons to about 40,000 Daltons, about 40,000 Daltons to about 50,000 Daltons, about 50,000 Daltons to about 60,000 Daltons, about 60,000 Daltons to about 100,000 Daltons, about 100,000 Daltons to about 150,000 Daltons or about 150,000 Daltons to about 200,000 Daltons. In a specific embodiment, the PVP provided herein has a weight average molecular weight of about 10,000 Daltons.

The weight average molecular weight of a polymer (e.g., PVP) disclosed herein can be determined using any methods known in the art. As a non-limiting example, in certain embodiments, the PVP provided herein can have a weight average molecular weight from about 2,000 Daltons to about 200,000 Daltons, wherein the weight average molecular weight is determined by an experimental method, for example, a light scattering methods. Light scattering methods for polymer characterization are known in the art (see, e.g., Zimm B. H. et al., Journal of Chemical Physics, (1948) 16(12):1099-1116). Without being bound by theory, when paired with size exclusion chromatography and a concentration detection method such as differential refractive index, light scattering detection can enable precise characterization of the molecular weight distribution of polydisperse polymer samples. See, for example, Wyatt P. J. et al., Analytica Chimica Acta, (1993) 272:1-40. The method further comprises deriving an average molecular weight from the polymer concentration and the molar mass. Methods for deriving average molecular weight from molar mass and concentration detection data are well known in the art and reviewed in, for example, Wyatt P. J. et al., Analytica Chimica Acta, (1993) 272:1-40.

In certain embodiments, the PVP disclosed herein is present in an amount of about 5% w/v to about 30% w/v of the aqueous composition. In certain embodiments, the PVP is present in about 5% w/v to about 6% w/v, about 6% w/v to about 7% w/v, about 7% w/v to about 8% w/v, about 8% w/v to about 9% w/v, about 9% w/v to about 10% w/v, about 10% w/v to about 11% w/v, about 11% w/v to about 12% w/v, about 12% w/v to about 13% w/v, about 13% w/v to about 14% w/v, about 14% w/v to about 15% w/v, about 15% w/v to about 16% w/v, about 16% w/v to about 17% w/v, about 17% w/v to about 18% w/v, about 18% w/v to about 19% w/v, about 19% w/v to about 20% w/v, about 20% w/v to about 21% w/v, about 21% w/v to about 22% w/v, about 22% w/v to about 23% w/v, about 23% w/v to about 24% w/v, about 24% w/v to about 25% w/v, about 25% w/v to about 26% w/v, about 26% w/v to about 27% w/v, about 27% w/v to about 28% w/v, about 28% w/v to about 29% w/v or about 29% w/v to about 30% w/v. In a specific embodiment, the PVP provided herein is present in an amount of about 15% w/v of the aqueous composition.

### 4.2.2 Apoptosis Inhibitor

In certain embodiments, the aqueous compositions disclosed herein comprise an apoptosis inhibitor. In certain embodiments, the apoptosis inhibitors disclosed herein is useful to prevent or inhibit apoptosis of cells in a biological sample, for example, a cell-containing biological sample (e.g., blood). Without being bound by theory, in certain embodiments, an apoptosis inhibitor disclosed herein is useful to preserve a cell-free nucleic acid population from changes in its composition, for example, arising from contamination with fragmented genomic DNA. In certain embodiments, the apoptosis inhibitor is useful to stabilize cells in a cell-containing biological sample. In certain embodiments, the apoptosis inhibitor is useful to inhibit degradation of nucleic acids (e.g., cfNA) in a cell-containing biological sample.

Any apoptosis inhibitor known in the art can be used in connection with the aqueous compositions disclosed herein. As a non-limiting example, apoptosis inhibitors can include compounds that act as metabolic inhibitors, inhibitors of nucleic acid degradation, enzyme inhibitors, caspase inhibitors, calpain inhibitors and inhibitors of other enzymes involved in apoptotic processes. In certain embodiments, the apoptosis inhibitor is cell-permeable.

In certain embodiments, one or more apoptosis inhibitors can be used. In certain embodiments, a combination of different apoptosis inhibitors, either from the same or a different class of apoptosis inhibitors can be used.

In certain embodiments, the apoptosis inhibitor is a caspase inhibitor, for example, a caspase inhibitor which acts upon one or more caspases located downstream in the intracellular apoptosis pathway of the cell, such as caspase-3. In certain embodiments, the caspase inhibitor can be an inhibitor of one or more caspases or caspase families (e.g., caspase-1, caspase-3, caspase-8, caspase-9, caspase-10 and caspase-12). In certain embodiments, a combination of caspase inhibitors can be used. In certain embodiments, the caspase inhibitor is a peptide inhibitor. In certain embodiments the caspase inhibitor can be a peptide that competes with caspase binding.

Without being bound by theory, reversible or irreversible inhibitors of caspase activation can be made by coupling caspase-specific peptides to compounds (e.g. aldehyde, nitrile or ketone compounds). As a non-limiting example, in certain embodiments, fluoromethyl ketone (FMK) derivatized peptides, such as methyl (3S)-5-fluoro-3-[[(2S)-2-[[(2S)-3-methyl-2-(phenylmethoxycarbonylamino)butanoyl]amino]propanoyl]amino]-4-oxopentanoate) (Z-VAD-FMK) can be used in the aqueous compositions disclosed herein. In certain embodiments, inhibitors made with a benzyloxycarbonyl group (BOC) at the N-terminus and O-methyl side chains can be used. In certain embodiments, caspase inhibitors can have a phenoxy group at the C-terminus, e.g., (3S)-5-(2,6-difluorophenoxy)-3-[[(2S)-3-methyl-2-(quinoline-2-carbonylamino)butanoyl]amino]-4-oxopentanoic acid) (Q-VD-OPh).

In certain embodiments, the apoptosis inhibitor is a “broad-spectrum” pan-caspase inhibitor (e.g., an inhibitor of multiple caspases). In certain embodiments, the caspase inhibitor may preferentially inhibit one or more particular caspases. In certain embodiments, an aqueous composition disclosed herein comprises an apoptosis inhibitor, wherein the apoptosis inhibitor is Q-VD-OPh ((3S)-5-(2,6-difluorophenoxy)-3-[[(2S)-3-methyl-2-(quinoline-2-carbonylamino)butanoyl]amino]-4-oxopentanoic acid), Z-VAD-FMK (methyl (3S)-5-fluoro-3-[[(2S)-2-[[(2S)-3-methyl-2-(phenylmethoxycarbonylamino)butanoyl]amino]propanoyl]amino]-4-oxopentanoate), Q-VD(OMe)-OPh ((S)-methyl 5-(2,6-difluorophenoxy)-3-((S)-3-methyl-2-(quinoline-2-carboxamido) butanamido)-4-oxopentanoate), or Boc-D-fmk (methyl 5-fluoro-3-[(2-methylpropan-2-yl) oxycarbonylamino]-4-oxopentanoate).

In certain embodiments, the apoptosis inhibitor is quinolyl-valyl-O-methylaspartyl-[-2,6-difluorophenoxy]-methyl ketone (Q-VD-OPh).

In certain embodiments, the apoptosis inhibitor is a derivative of an apoptosis inhibitor disclosed herein or known in the art. As a non-limiting example, in certain embodiments the apoptosis inhibitor can be a derivative of Q-VD-OPh, (e.g., O-methylated Q-VD-OPh).

In certain embodiments, the Q-VD-OPh has a concentration of between about 50 μM and about 5000 μM. In certain embodiments, the Q-VD-OPh has a concentration of between about 50 μM and about 100 μM, about 100 μM and about 200 μM, about 200 μM and about 300 μM, about 300 μM and about 400 μM, about 400 μM and about 500 μM, about 500 μM and about 600 μM, about 600 μM and about 700 μM, about 700 μM and about 800 μM, about 800 μM and about 900 μM, about 900 μM and about 1000 μM, about 1000 μM and about 1100 μM, about 1100 μM and about 1200 μM, about 1200 μM and about 1300 μM, about 1300 μM and about 1400 μM, about 1400 μM and about 1500 μM, about 1500 μM and about 1600 μM, about 1600 μM and about 1700 μM, about 1700 μM and about 1800 μM, about 1800 μM and about 1900 μM, about 1900 μM and about 2000 μM, about 2000 μM and about 2500 μM, about 2500 μM and about 3000 μM, about 3000 μM and about 35000 μM, about 3500 μM and about 4000 μM, about 4000 μM and about 4500 μM or about 4500 μM and about 5000 μM. In a specific embodiment, the Q-VD-OPh has a concentration of about 550 μM.

### 4.2.3 Eryptosis Inhibitors

In certain embodiments, the aqueous compositions disclosed herein comprise one or more eryptosis inhibitors.

Without being bound by theory, eryptosis can be triggered by a range of signals, including energy depletion, hyperosmotic shock, oxidative stress, increased cytosolic Ca2+ activity ([Ca2+]i) and xenobiotics (Huber S. M. et al., Pflugers Arch. (2001) 441(4):551-8; Lang K. S. et al., Cell Death Differ. (2004) 11(2):231-43; Klarl B. A. et al., Am J Physiol Cell Physiol. (2006) 290(1):C244-53; Lang F. et al., Antioxid Redox Signal. (2014) 21(1):138-53; Lang E. and Lang F., Biomed Res Int. (2015) 513518; and Pretorius E., et al., Cell Physiol Biochem. (2016) 39(5):1977-2000).

The cellular mechanisms underlying eryptosis include an increase of cytosolic Ca2+ activity ([Ca2+]i) (Lang E and Lang F. Semin Cell Dev Biol (2015) 39: 35-42), ceramide (Abed M. et al., Am J Physiol Cell Physiol (2012) 303:C991-999; Lang E. et al., Apoptosis (2015) 5:758-767), G-protein Galphai2 (Bissinger R. et al., Sci Rep (2016) 6: 30925), and activation of diverse kinases including casein kinase 1α, Janus-activated kinase JAK3, protein kinase C, and p38 kinase (Lang E and Lang F. Semin Cell Dev Biol (2015) 39: 35-42). Eryptosis is inhibited by several kinases including AMP activated kinase AMPK, cGMP-dependent protein kinase, PAK2 kinase, and mitogen and stress activated kinase MSK1/2 (Lang E. et al., Sci Rep (2015) 5:17316).

Any eryptosis inhibitor known in the art can be used in connection with the aqueous compositions disclosed herein. In certain embodiments, the eryptosis inhibitor is an inhibitor of an eryptosis signaling pathway or a molecular or cellular mechanism underlying eryptosis disclosed herein or known in the art. As a non-limiting example, in certain embodiments, an eryptosis inhibitor can be an inhibitor of increase of cytosolic Ca2+ activity (e.g., an inhibitor of Ca2+ entry into cells). In certain embodiments, an eryptosis inhibitor can be an inhibitor of ceramide formation. In certain embodiments, an eryptosis inhibitor can be an inhibitor of ATP depletion.

Non-limiting examples of eryptosis inhibitors include adenosine, amitriptyline, caffeine, a catecholamine, D4476, dibutyryl-cGMP, dithiothreitol, ethylisopropylamiloride, erythropoietin, flufenamic acid, furosemide, glutathione, 7-monohydroxyethylrutoside, N-acetylcysteine, naringin, 1,2,3,4-tetrahydro-6-nitro-2,3-dioxo-benzo[f]quinoxaline-7-sulfonamide, 6-cyano-7-nitroquinoxaline-2,3-dione (CNQX), niflumic acid, nitroprusside, 5-nitro-2-(3-phenylpropylamino)benzoic acid (NPPB), papanonoate, P38 Inh III, resveratrol, (R)-DRF503, salidroside, 4-(4-fluorophenyl)-2-(4-methylsulfinylphenyl)-5-(4-pyridyl)imidazole (SB203580), Staurosporine, Trolox, urea, vitamin E, xanthohumol, and Zidovudine.

### 4.2.3.1 Naringin

In certain embodiments, the aqueous compositions disclosed herein comprise naringin. Naringin, also known as naringoside and naringin hydrate, is a disaccharide derivative that is (S)-naringenin substituted by a 2-O-(alpha-L-rhamnopyranosyl)-beta-D-glucopyranosyl moiety at position 7 via a glycosidic linkage. The chemical name for naringin is (2S)-7-[(2S,3R,4S,5S,6R)-4,5-dihydroxy-6-(hydroxymethyl)-3-[(2S,3R,4R,5R,6S)-3,4,5-trihydroxy-6-methyloxan-2-yl]oxyoxan-2-yl]oxy-5-hydroxy-2-(4-hydroxyphenyl)-2,3-dihydrochromen-4-one.

In certain embodiments, naringin has a concentration of between about 40 nM and about 40 μM. In certain embodiments, naringin has a concentration of between about 40 nM and about 100 nM, about 100 nM and about 200 nM, about 200 nM and about 300 nM, about 300 nM and about 400 nM, about 400 nM and about 500 nM, about 500 nM and about 600 nM, about 600 nM and about 700 nM, about 700 nM and about 800 nM, about 800 nM and about 900 nM, about 900 nM and about 1 μM, about 1 μM and about 2 μM, about 2 μM and about 3 μM, about 3 μM and about 4 μM, about 4 μM and about 5 μM, about 5 μM and about 10 μM, about 10 μM and about 15 μM, about 15 μM and about 20 μM, about 20 μM and about 25 μM, about 25 μM and about 30 μM, about 30 μM and about 35 μM or about 35 μM and about 40 μM. In a specific embodiment, naringin has a concentration of about 400 nM.

### 4.2.3.2 Urea

In certain embodiments, the aqueous compositions disclosed herein comprise urea. In certain embodiments, the urea has a concentration of between about 4 mM and about 650 mM. In certain embodiments, the urea has a concentration of between about 4 mM and about 8 mM, about 8 mM and about 12 mM, about 12 mM and about 16 mM, about 16 mM and about 20 mM, about 20 mM and about 24 mM, about 24 mM and about 28 mM, about 28 mM and about 32 mM, about 32 mM and about 36 mM, about 36 mM and about 40 mM, about 40 mM and about 44 mM, about 44 mM and about 48 mM, about 48 mM and about 52 mM, about 52 mM and about 56 mM, about 56 mM and about 60 mM, about 60 mM and about 64 mM, about 64 mM and about 68 mM, about 68 mM and about 72 mM, about 72 mM and about 76 mM, about 76 mM and about 80 mM, about 80 mM and about 84 mM, about 84 mM and about 88 mM, about 88 mM and about 92 mM, about 92 mM and about 96 mM, about 96 mM and about 100 mM, about 100 mM and about 150 mM, about 150 mM and about 200 mM, about 200 mM and about 250 mM, about 250 mM and about 300 mM, about 300 mM and about 350 mM, about 350 mM and about 400 mM, about 400 mM and about 450 mM, about 450 mM and about 500 mM, about 500 mM and about 550 mM, about 550 mM and about 600 mM or about 600 mM and about 650 mM. In a specific embodiment, the urea has a concentration of about 40 mM.

### 4.2.3.3 Caffeine

In certain embodiments, the aqueous compositions disclosed herein comprise caffeine. In certain embodiments, the caffeine has a concentration of between about 2 μM and about 500 μM. In certain embodiments, the caffeine has a concentration of between about 2 μM and about 4 μM, about 4 μM and about 6 μM, about 6 μM and about 8 μM, about 8 μM and about 10 μM, about 10 μM and about 12 μM, about 12 μM and about 14 μM, about 14 μM and about 16 μM, about 16 μM and about 18 μM, about 18 μM and about 20 μM, about 20 μM and about 22 μM, about 22 μM and about 24 μM, about 24 μM and about 26 μM, about 26 μM and about 28 μM, about 28 μM and about 30 μM, about 30 μM and about 32 μM, about 32 μM and about 34 μM, about 34 μM and about 36 μM, about 36 μM and about 38 μM, about 38 μM and about 40 μM, about 40 μM and about 42 μM, about 42 μM and about 44 μM, about 44 μM and about 46 μM, about 46 μM and about 48 μM, about 48 μM and about 50 μM, about 50 μM and about 100 μM, about 100 μM and about 150 μM, about 150 μM and about 200 μM, about 200 μM and about 250 μM, about 250 μM and about 300 μM, about 300 μM and about 350 μM, about 350 μM and about 400 μM or about 450 μM and about 500 μM. In a specific embodiment, the caffeine has a concentration of about 20 μM.

### 4.2.4 Polyethylene Glycol Dimethyl Ether

In certain embodiments, the aqueous compositions disclosed herein comprise polyethylene glycol dimethyl ether (dmPEG).

In certain embodiments, the weight average molecular weight of the dmPEG provided herein can be from about 250 Daltons to about 4,000 Daltons (g/mol). In certain embodiments, the dmPEG has a weight average molecular weight of about 250 Daltons to about 500 Daltons, about 500 Daltons to about 750 Daltons, about 750 Daltons to about 1,000 Daltons, about 1,000 Daltons to about 1,250 Daltons, about 1,250 Daltons to about 1,500 Daltons, about 1,500 Daltons to about 1,750 Daltons, about 1,750 Daltons to about 2,000 Daltons, about 2,000 Daltons to about 2,250 Daltons, about 2,250 Daltons to about 2,500 Daltons, about 2,500 Daltons to about 2,750 Daltons, about 2,750 Daltons to about 3,000 Daltons, about 3,250 Daltons to about 3,500 Daltons, about 3,500 Daltons to about 3,750 Daltons, about 3,750 Daltons to about 4,000 Daltons, about 4,000 Daltons to about 4,250 Daltons, about 4,250 Daltons to about 4,500 Daltons, about 4,500 Daltons to about 4,750 Daltons or about 4,750 Daltons to about 5,000 Daltons. In a specific embodiment, the dmPEG provided herein has a weight average molecular weight of about 2,000 Daltons.

In certain embodiments, the dmPEG disclosed herein is present in an amount of about 5% w/v to about 30% w/v of the aqueous composition. In certain embodiments, the dmPEG is present in about 5% w/v to about 6% w/v, about 6% w/v to about 7% w/v, about 7% w/v to about 8% w/v, about 8% w/v to about 9% w/v, about 9% w/v to about 10% w/v, about 10% w/v to about 11% w/v, about 11% w/v to about 12% w/v, about 12% w/v to about 13% w/v, about 13% w/v to about 14% w/v, about 14% w/v to about 15% w/v, about 15% w/v to about 16% w/v, about 16% w/v to about 17% w/v, about 17% w/v to about 18% w/v, about 18% w/v to about 19% w/v, about 19% w/v to about 20% w/v, about 20% w/v to about 21% w/v, about 21% w/v to about 22% w/v, about 22% w/v to about 23% w/v, about 23% w/v to about 24% w/v, about 24% w/v to about 25% w/v, about 25% w/v to about 26% w/v, about 26% w/v to about 27% w/v, about 27% w/v to about 28% w/v, about 28% w/v to about 29% w/v or about 29% w/v to about 30% w/v. In a specific embodiment, the dmPEG provided herein is present in an amount of about 15% w/v of the aqueous composition.

### 4.2.5 Aqueous and Saline Solutions

In certain embodiments, the aqueous compositions disclosed herein are provided as an aqueous solution. In certain embodiments, the aqueous solution is a saline solution, for example, an aqueous solution which comprises one or more salts such as, e.g., KCl, NaCl, and the like. In certain embodiments, an aqueous composition disclosed herein is useful to maintain an isotonic environment in a biological sample, for example, a cell-containing biological sample (e.g., blood). For example, in certain embodiments an aqueous composition disclosed herein is useful to maintain an isotonic environment so that cells neither shrink nor swell. In certain embodiments, the aqueous composition is useful to maintain an osmotic balance (e.g., maintain a salt and water balance across a biological membrane). In certain embodiments, the aqueous composition is useful to maintain a desired extracellular osmolarity.

In certain embodiments, the aqueous solution may comprise KCl. In certain embodiments, the KCl has a concentration of between about 0.1 mM to about 0.6 mM. In certain embodiments, the KCl has a concentration of between about 0.1 mM to about 0.15 mM, about 0.15 mM to about 0.2 mM, about 0.2 mM to about 0.25 mM, about 0.25 mM to about 0.3 mM, about 0.3 mM to about 0.35 mM, about 0.35 mM to about 0.4 mM, about 0.4 mM to about 0.45 mM, about 0.45 mM to about 0.5 mM, about 0.5 mM to about 0.55 mM, or about 0.55 mM to about 0.6 mM. In a specific embodiment, the KCl has a concentration of about 0.27 mM.

In certain embodiments, the aqueous solution may comprise NaCl. In certain embodiments, the NaCl has a concentration of between about 6.5 mM to about 27.5 mM. In certain embodiments, the NaCl has a concentration of between about 6.5 mM to about 8.5 mM, about 8.5 mM to about 10.5 mM, about 10.5 mM to about 12.5 mM, about 12.5 mM to about 14.5 mM, about 14.5 mM to about 16.5 mM, about 16.5 mM to about 18.5 mM, about 18.5 mM to about 20.5 mM, about 20.5 mM to about 22.5 mM, about 22.5 mM to about 24.5 mM, about 24.5 mM to about 26.5 mM or about 26.5 mM to about 27.5 mM. In a specific embodiment, the NaCl has a concentration of about 13.7 mM.

### 4.2.6 Anticoagulant

In certain embodiments, the aqueous compositions disclosed herein comprise an anticoagulant. In certain embodiments, the anticoagulant is useful to inhibit enzymatic reactions, e.g., in a blood sample, and prevent separation of the cellular and cell-free fractions. In certain embodiments, the anticoagulant is a chelating reagent. In certain embodiments, the chelating agent chelates divalent metal ions (e.g., divalent metal ions required for nuclease enzyme activity).

Any chelator known in the art can be used. As a non-limiting example, in certain embodiments, the chelator can be ethylenediamine tetraacetic acid (EDTA) and any salts thereof, cyclohexane diaminetetraacetate (CDTA), diethylenetriamine pentaacetic acid (DTPA), tetraazacyclododecanetetraacetic acid (DOTA), tetraazacyclotetradecanetetraacetic acid (TETA), and desferrioximine, ethylene glycol tetraacetic acid (EGTA), hydroxyethylethylenediaminetriacetic acid (HEDTA), N,N-bis(carboxymethyl)glycine (NTA), citrate anhydrous, sodium citrate, calcium citrate, ammonium citrate, ammonium bicitrate, citric acid, diammonium citrate, potassium citrate, magnesium citrate, ferric ammonium citrate, lithium citrate or chelator analogs thereof. For example, blood samples can be collected in blood collection tubes containing spray-dried or liquid EDTA. In certain embodiments, the anticoagulant is selected from the group consisting of heparin, ethylenediamine tetraacetic acid (EDTA), citrate, oxalate, K₂EDTA, K₃EDTA, or any combination thereof.

### 4.2.7 Sugar

In certain embodiments, the aqueous compositions disclosed herein comprises an energy source, for example, a sugar.

Without being bound by theory, the addition of an energy source (e.g., sugar) to an aqueous composition disclosed herein is useful to prevent cellular stress from starvation. In certain embodiments, the aqueous composition disclosed herein can comprise one or more sugars. As a non-limiting example, in certain embodiments, an aqueous composition disclosed herein can comprise glucose, lactose, fructose, galactose, trehalose, sucrose, mannitol, galactose, mannose, sorbitol, maltose and/or ribose.

In certain embodiments, an aqueous composition disclosed herein comprises glucose and the glucose has a concentration of between about 10 mM to about 100 mM. In certain embodiments, glucose has a concentration of about 10 mM to about 50 mM, about 50 mM to about 100 mM, about 100 mM to about 150 mM, about 150 mM to about 200 mM, about 200 mM to about 250 mM, about 250 mM to about 300 mM, about 300 mM to about 350 mM, about 350 mM to about 400 mM, about 400 mM to about 450 mM, about 500 mM to about 550 mM, about 550 mM to about 600 mM, about 600 mM to about 650 mM, about 650 mM to about 700 mM, about 700 mM to about 750 mM, about 750 mM to about 800 mM, about 800 mM to about 850 mM, about 850 mM to about 900 mM, about 900 mM to about 950 mM or about 950 mM to about 1000 mM. In a specific embodiment, an aqueous composition disclosed herein comprises glucose at a concentration of about 100 mM.

### 4.2.8 Additional Agents

In certain embodiments, the aqueous compositions disclosed herein comprise one or more additional agents. As a non-limiting example, in certain embodiments, the one or more additional agents can be antibiotics, preservatives, surfactants, antioxidants, mold inhibitors, nucleic acids, pH adjusters, osmolarity adjusters, nuclease inhibitors, protease inhibitors or any combination thereof.

In certain embodiments, the aqueous composition disclosed herein can comprise one or more nuclease inhibitors. As an non-limiting example, in certain embodiments, the nuclease inhibitor can be diethyl pyrocarbonate, ethanol, aurintricarboxylic acid (ATA), formamide, vanadyl-ribonucleoside complexes, macaloid, ethylenediamine tetraacetic acid (EDTA), proteinase K, heparin, hydroxylamine-oxygen-cupric ion, bentonite, ammonium sulfate, dithiothreitol (DTT), beta-mercaptoethanol, cysteine, dithioerythritol, tris(2-carboxyethyl) phosphene hydrochloride, a divalent cation such as Mg²⁺, Mn²⁺, Zn²⁺, Fe²⁺, Ca²⁺, Cu²⁺, or any combination thereof.

### 4.2.9 pH

The pH of the aqueous compositions disclosed herein can between about 5.0 to about 11.0. In certain embodiments, the pH can be between about 5 to about 5.5, about 5.5 to about 6, about 6 to about 6.5, about 6.5 to about 7, about 7 to about 7.5, about 7.5 to about 8, about 8 to about 8.5, about 8.5 to about 9, about 9 to about 9.5, about 9.5 to about 10, about 10 to about 10.5, or about 10.5 to about 11. In a specific embodiment, the pH can be between about 8 to about 8.5. In certain embodiments, a buffer, such as HEPES, TRIS, BES or carbonate buffer can be added to the aqueous composition to maintain the pH in a constant range.

### 4.2.10 Biological Aspects

In certain embodiments, the aqueous compositions disclosed herein stabilize a cfNA population in a biological sample, for example, a cell-containing biological sample (e.g., blood).e.g. In certain embodiments, an aqueous composition disclosed herein is useful to preserve, protect and/or maintain the integrity of one or a plurality of cfNAs and/or cfNA populations. In certain embodiments, an aqueous composition disclosed herein is useful to inhibit and/or reduce the fragmentation, degradation and/or modification of one or a plurality of cfNAs or cfNA populations that are present in the biological sample. In certain embodiments, an aqueous composition disclosed herein is useful to stabilize a cfNA and/or cfNA population such that it will be of sufficient quality and/or quantity for subsequent processes such as amplification, hybridization or sequencing, following storage and/or incubation at, e.g., ambient temperature, e.g., for a desired amount of time.

In certain embodiments, a cfNA population in a sample (e.g., a blood sample) is stabilized by any desired amount. As a non-limiting example, a composition, kit, and/or method of the present disclosure may be used to stabilize one or more cfNAs within a sample by 50% (e.g., the degradation and/or fragmentation rate of the cfNA may be reduced by about 50%). The stabilization efficiency for any compositions, kits, and/or methods of the present disclosure may be greater than about 10%, about 15%, about 20%, about 25%, about 30%, about 40%, about 50%, about 60%, about 70%, about 80%, about 90%, about 100%, or greater than about 100% for the desired amount of time. In certain embodiments, the stabilization efficiency may fall within a range. For example, the stabilization efficiency for a composition, kit, and/or method of the present disclosure may be between about 10% and about 40% for a desired amount of time. The stabilization and/or stabilization efficiency provided by a composition, kit, and/or method provided herein can be measured or analyzed using any methods disclosed herein or known in the art. As a non-limiting example, stabilization and/or stabilization efficiency can be determined using a hemolysis assay, gel electrophoresis of nucleic acids (e.g., cfDNA, cfRNA and/or gDNA), PCR techniques (e.g., rtPCR, qPCR, rt-qPCR), apoptosis assays, eryptosis assays, or flow cytometry. In some embodiments, the stabilization and/or stabilization efficiency can be determined using any method known in the art to quantify nucleic acids (e.g., DNA and/or RNA) or to assay the integrity and/or quality of nucleic acids. As a non-limiting example, in some embodiments, the stabilization and/or stabilization efficiency can be determined by fluorometric quantification of nucleic acids (e.g., Qubit Fluorometric Quantitiation, Thermo Fisher Scientific).

In certain embodiments, the aqueous compositions disclosed herein are useful to minimize, reduce or inhibit cellular lysis in an aqueous solution (e.g., blood). In certain embodiments, the aqueous compositions disclosed herein are useful to minimize, reduce or inhibit lysis of erythrocytes (red blood cells) in a sample. In certain embodiments, the aqueous compositions disclosed herein are useful to minimize, reduce or inhibit lysis of leukocytes (white blood cells). In certain embodiments, the lysis of any populations or subpopulations of cells in a sample is minimized, reduced or inhibited. For instance, in certain embodiments, the aqueous compositions disclosed herein are useful to inhibit lysis of one or more multiple subpopulations of white blood cells in a sample

It will be understood that the detection and analysis of cell-free nucleic acids such as DNA and/or RNA can provide a useful tool for the detection, screening, diagnosis, prognosis, and monitoring of medical conditions, diseases and infectious processes. cfNA can also be analyzed to identify potential therapeutic targets and monitor treatment response. Exemplary applications and analysis methods of cfNA are e.g. described in WO97/035589, WO97/34015, Swamp V. et al, FEBS Letters (2007) 581(5):795-799; Fleischhacker M. et al., Ann. N.Y. Acad. Sci. (2006) 1075:40-49; Fleischhacker M. and Schmidt, Biochmica et Biophysica Acta (2007) 1775:191 -232; Hromadnikova I. et al DNA and Cell Biology, (2006) 25(11):635-640; Fan H. C. et al., Clinical Chemistry (2010) 56(8):1279-1286.

In certain embodiments, the aqueous compositions disclosed herein can be used to stabilize a blood sample. In certain embodiments, the aqueous compositions disclosed herein can be used to stabilize a blood sample prior to any processing or analysis methods known in the art. In certain embodiments, the aqueous compositions disclosed herein can be used to stabilize a processed blood sample (e.g., serum or plasma). One of skill in the art would consider that a “cell-free” processed blood sample, such as plasma or serum, may contain residual cells that were not removed during processing. In certain embodiments, the aqueous compositions disclosed herein can be used to stabilize a plasma or serum sample.

### 4.2.11 cfNA

In certain embodiments, the cfNA is derived from a biological sample at the site of a disease (e.g., a tumor). In certain embodiments, the cfNA is derived from a biological sample at the site of a pre-malignant disease.

It will be understood that maternal plasma cfDNA pattern may be used to determine fetus gender identity and may also allow detection of inherited and de novo monogenic mutations. Accordingly, it will be understood that cfNA can represent possible disease-related genes or genetic variants derived from a subject or a fetus. In certain embodiments, the cfNA is derived from a biological sample comprising fetal DNA.

In certain embodiments, the cfNA is derived from a biological sample that comprises pathogenic DNA (e.g., bacterial or viral DNA or RNA). For example, in certain embodiments, the aqueous compositions disclosed herein are useful to monitor viral load.

### 4.2.12 Effect on Cells

In certain embodiments, the aqueous compositions disclosed herein are useful to reduce or inhibit cellular damage, aging or death in a biological sample, for example, a cell-containing biological sample (e.g., blood). In certain embodiments, the aqueous compositions disclosed herein are useful to reduce or inhibit cell lysis in a biological sample, for example, a cell-containing biological sample (e.g., blood). In certain embodiments, the aqueous compositions disclosed herein are useful to reduce or inhibit apoptosis or eryptosis in a biological sample, for example, a cell-containing biological sample (e.g., blood). In certain embodiments, the aqueous compositions disclosed herein are useful to increase cell life span in a biological sample, for example, a cell-containing biological sample (e.g., blood).

In certain embodiments, the aqueous compositions disclosed herein are useful to reduce or inhibit changes in cellular membranes in a biological sample, for example, a cell-containing biological sample (e.g., blood). In certain embodiments, the aqueous compositions disclosed herein are useful to reduce or inhibit changes in cell susceptibility to recognition by macrophages in a biological sample, for example, a cell-containing biological sample (e.g., blood). In certain embodiments, the aqueous compositions disclosed herein are useful to reduce or inhibit phagocytosis of cells in a biological sample, for example, a cell-containing biological sample (e.g., blood). In certain embodiments, the aqueous compositions disclosed herein are useful to reduce or inhibit erythrophagocytosis in a biological sample, for example, a cell-containing biological sample (e.g., blood). In certain embodiments, the aqueous compositions disclosed herein are useful to reduce or inhibit oxidative damage of cells in a biological sample, for example, a cell-containing biological sample (e.g., blood). In certain embodiments, the aqueous compositions disclosed herein are useful to reduce or inhibit cell destruction in a biological sample, for example, a cell-containing biological sample (e.g., blood).

In certain embodiments, the aqueous compositions disclosed herein are useful to promote cellular homeostasis in a biological sample, for example, a cell-containing biological sample (e.g., blood). In certain embodiments, the aqueous compositions disclosed herein are useful to promote cellular longevity in a biological sample, for example, a cell-containing biological sample (e.g., blood).

In certain embodiments, the aqueous compositions disclosed herein are useful to prevent, reduce or inhibit cellular apoptosis in a biological sample, for example, a cell-containing biological sample (e.g., blood).

### 4.2.13 Effect on Nucleic Acids

In certain embodiments, the aqueous compositions disclosed herein are useful to prevent or inhibit the release of nucleic acids from cells in a biological sample, for example, a cell-containing biological sample (e.g., blood). In certain embodiments, the aqueous compositions disclosed herein are useful to prevent or inhibit the release of genomic (nuclear) DNA from cells in a biological sample, for example, a cell-containing biological sample (e.g., blood). In certain embodiments, the aqueous compositions disclosed herein are useful to prevent or inhibit the release of RNA (e.g., cytoplasmic RNA, mitochondrial RNA, miRNAs) from cells in a biological sample, for example, a cell-containing biological sample (e.g., blood). In certain embodiments, the aqueous compositions disclosed herein are useful to prevent or inhibit the contamination of cfNA with cellular nucleic acids (e.g., nucleic acids released from cells) in a biological sample, for example, a cell-containing biological sample (e.g., blood). In certain embodiments, the aqueous compositions disclosed herein are useful to prevent or inhibit the contamination of cfNA with fragmented cellular nucleic acids in a biological sample, for example, a cell-containing biological sample (e.g., blood). In certain embodiments, the aqueous compositions disclosed herein are useful to prevent or inhibit dilution of cfNA with cellular nucleic acids in a biological sample, for example, a cell-containing biological sample (e.g., blood).

When resolved using electrophoresis on agarose gels, apoptotic DNA initially has a characteristic “ladder” pattern, as opposed to a smear of nucleic acids that is observed, for example, in necrosis or other non-specific DNA degradation. In certain embodiments, the aqueous compositions disclosed herein are useful to prevent, reduce or inhibit the fragmentation of cellular DNA (e.g., genomic DNA) in a biological sample, for example, a cell-containing biological sample (e.g., blood). In certain embodiments, the aqueous compositions disclosed herein are useful to prevent, reduce or inhibit the fragmentation of DNA resulting in the observation of an apoptotic “ladder” pattern in a biological sample, for example, a cell-containing biological sample (e.g., blood).

In certain embodiments, the aqueous compositions disclosed herein are useful to prevent or inhibit release or spillage of genomic DNA from white blood cells in a biological sample, for example, a cell-containing biological sample (e.g., blood).

In certain embodiments, the aqueous compositions disclosed herein are useful to prevent or inhibit release or spillage of cellular RNA from white blood cells in a biological sample, for example, a cell-containing biological sample (e.g., blood). In certain embodiments, the aqueous compositions disclosed herein are useful to prevent or inhibit release or spillage of cellular RNA from red blood cells in a biological sample, for example, a cell-containing biological sample (e.g., blood). In certain embodiments, the aqueous compositions disclosed herein are useful to maintain or promote the structural integrity of cfNA in a biological sample, for example, a cell-containing biological sample (e.g., blood).

### 4.2.14 Methods for Stabilization

Also provided herein are methods for stabilizing a cell-free nucleic acid (cfNA) population in a cell-containing biological sample (e.g., whole blood) comprising contacting the cell-containing biological sample with an aqueous composition disclosed herein. In certain embodiments, the methods are for stabilizing a cell-free nucleic acid (cfNA) population in a cell-containing biological sample (e.g., whole blood) comprising contacting the cell-containing biological sample with an aqueous composition disclosed herein, wherein the resulting mixture comprises a ratio of the cell-containing biological sample to the aqueous composition of about 6:1 to about 12:1. In certain embodiments, the resulting mixture comprises a ratio of the cell-containing biological sample to the aqueous composition of about 9:1.

In certain embodiments, the methods comprise stabilizing a cell-free nucleic acid (cfNA) population in a cell-containing biological sample, wherein the cell-containing biological sample comprises cell-free DNA and/or cell-free RNA.

In certain embodiments, the methods further comprise storing the resulting mixture for about one hour to about ten days. In certain embodiments, the resulting mixture is stored between about 1 hour and about 6 hours, about 6 hours and about 12 hours, about 12 hours and about 18 hours, about 18 hours and about 24 hours (1 day), about 1 day and about 2 days, about 2 days and about 3 days, about 3 days and about 4 days, about 4 days and about 5 days, about 5 days and about 6 days, about 6 days and about 7 days, about 7 days and about 8 days, about 8 days and about 9 days or about 9 days and about 10 days. In certain embodiments, the resulting mixture is stored for more than about 10 days. In a specific embodiment, the resulting mixture is stored for about five days or longer.

In certain embodiments, the methods further comprise storing the resulting mixture at ambient temperature. In certain embodiments, the methods further comprise storing the resulting mixture at room temperature. In certain embodiments, the methods further comprise storing the resulting mixture at a temperature above about 4° C. (e.g., without refrigeration). One of skill in the art will appreciate that samples stored at ambient temperature or room temperature may undergo variations in temperature during storage and/or transport. In certain embodiments, the methods further comprise storing the resulting mixture at a temperature of about 18° C. to about 30° C. In certain embodiments, the resulting mixture is stored between about 18° C. to about 20° C., about 20° C. to about 20° C., about 20° C. to about 22° C., about 22° C. to about 24° C., about 24° C. to about 26° C., about 26° C. to about 28° C. or about 28° C. to about 30° C. In certain embodiments, the resulting mixture is stored at a temperature greater than 30° C. In certain embodiments, the resulting mixture is stored at a temperature less than about 18° C., less than about 10° C., or less than about 4° C. In certain embodiments, the methods disclosed herein allow storage and/or transport of cell-containing biological samples at about 4° C. (e.g., with refrigeration).

Optionally, the methods disclosed herein include contacting the cell-containing biological sample with an aqueous composition disclosed herein, wherein the resulting mixture is mixed (e.g., by inversion) about 1 time to about 12 times. In certain embodiments, the resulting mixture is mixed about 1 time to about 3 times, about 3 times to about 6 times, about 6 times to about 9 times, or about 9 times to about 12 times. In a specific embodiment, the resulting mixture is mixed (e.g., by inversion) about 10 times.

One of skill in the art will appreciate that the methods disclosed herein allow storage and/or transport of cell-containing biological samples (e.g., blood samples) while stabilizing cfNA populations within the sample. For example, in certain embodiments, the aqueous compositions and methods disclosed herein stabilize cfNA populations in a blood sample during transport of samples to a facility for the processing, storage, testing and analysis of the samples. In certain embodiments, the sample is blood drawn from a subject. In certain embodiments, the blood is stored in a collection container or container (e.g., a vacuum blood tube) during storage or transport. The collection container can be any blood collection container known in the art (e.g., sterile blood collection evacuated tubes).

In certain embodiments, the sample (e.g., blood) is added to the collection container (e.g., the blood collection tube) prior to, concurrently with, and/or subsequently to the addition of an aqueous composition disclosed herein. In certain embodiments, the collection container (e.g., the blood collection tube) contains an aqueous composition disclosed herein. In certain embodiments, the collection container contains an aqueous composition disclosed herein, and the sample (e.g., blood) is subsequently added to the collection container (e.g., the blood collection tube).

### 4.2.15 Kit

Also provided herein is a kit comprising one or more of the aqueous compositions disclosed herein. In certain embodiments, the kit is useful for the stabilization of a cfNA population in a blood sample. As a non-limiting example, in certain embodiments, the kit is useful for the stabilize a cfNA population in a blood sample stored at a desired temperature (e.g., ambient temperature) for a desired amount of time (e.g., five or more days). In certain embodiments, the kit comprises a blood collection container and instructional materials. In certain embodiments, the kit comprises at least one aqueous composition disclosed herein in one or more containers. In certain embodiments, the kit contains all of the components necessary and/or sufficient to perform stabilize a cfNA population as disclosed herein, including all controls, instructions, and any necessary components for incubation of samples, transport of samples, and/or analysis of samples. In certain embodiments, the kit contains additional components for further processing of samples. One skilled in the art will readily recognize that the disclosed aqueous compositions of the present disclosure can be readily incorporated into one of the established kit formats which are well known in the art.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### 5. EXAMPLES

### 5.1 General Methods

### 5.1.1 Method 1: Separation of Blood Plasma

To separate plasma from whole blood, freshly-drawn whole blood samples were centrifuged at 1,900×g for 15 minutes at room temperature. The plasma fraction was removed and centrifuged again at 12,000×g for 15 minutes at room temperature. The resulting double-centrifuged plasma samples were assessed for hemolysis by measuring UV-Vis absorbance.

### 5.1.2 Method 2: Analysis of Blood Hemolysis in Plasma

To assess plasma samples separated from whole blood for hemolysis, 2 μl of each plasma sample was loaded to a UV-Vis spectrophotometer (NanoDrop™ 2000, Thermo Scientific). Absorbance was measured at 412 nm (A₄₁₂) or 415 nm (A₄₁₅) to determine the relative amount of free heme released from lysed red blood cells.

### 5.1.3 Method 3: Cell-Free DNA Purification

Cell-free DNA was purified from separated plasma samples using the Quick-cfDNA Serum & Plasma Kit per the manufacturer's instructions (Cat. No. D4076, Zymo Research Corp). Briefly, digestion buffer and proteinase K were added to plasma samples, which were mixed and then digested at 55° C. for 30 minutes. Following digestion, binding buffer was added, samples were mixed, and ethanol was added. Sample lysates were passed through a silica-membrane column and additional wash and concentration steps were performed. cfDNA was eluted in 35 μl elution buffer. Purified cfDNA obtained from plasma samples was stored at −20° C.

### 5.1.4 Method 4: Microcapillary Gel Electrophoresis Analysis of DNA

Microcapillary gel electrophoresis was used to perform a qualitative assessment of DNA size distribution and amount using DNA High Sensitivity Tapes (2100 TapeStation; Agilent Technologies). The manufacturer's suggested protocol was followed. Briefly, 2 μl of the eluate was mixed with 2 μl of the dye-containing buffer, and run on the tape in the TapeStation electrophoresis machine. A molecular weight marker was run in lane 1 (ladder).

### 5.1.5 Method 5: Quantitative PCR Analysis of DNA

Quantitative PCR of a fragment of the human satellite 2 (Sat2) locus. The Sat2 locus fragment is in an inactive state in most cells, remaining tightly bound to histones, which protect the bound DNA from degradation in blood once released from dead cells. The resulting PCR product size was 196 base pairs (bp). The forward and reverse primer sequences were 5′ CAT CAA TCG GAT GGA AAC GAA TGG 3′ (SEQ ID NO:1) and 5′ TTC GCG ACC ATT GGA TGA TTG CAG 3′ (SEQ ID NO:2), respectively. 10 μl PCR reactions were prepared with 1 μM of each primer, 3 μl of the sample eluate and 5 μl ZymoTaq 2×PCR master-mix (Cat. No. E2004, Zymo Research). The following cycle condition was used for PCR: 95° C. for 10 min, 35 cycles of: 95° C. for 30 sec, 58° C. for 30 sec, 72° C. for 1 min. Quantitative PCR reactions were run on CFX96 Touch™ Real-Time PCR Detection System (Model No. 1855195, Bio-Rad Laboratories, Inc.).

The average of duplicates for each sample was used to obtain the cycle threshold (Ct) value. The Ct value is the PCR cycle number at which the sample's reaction curve intersects the threshold line, the level of detection at which a reaction reaches a fluorescence intensity above background levels. The Ct value indicates how many cycles are required to detect a real signal from the sample. Ct values are inversely correlated to the amount of target nucleic acid in the sample.

### 5.1.6 Method 6: Cell-Free RNA Purification

Cell-free RNA was purified from separated plasma samples using the Quick-cfDNA/cfRNA Serum & Plasma Kit per the manufacturer's instructions (Cat. No. R1072, Zymo Research Corp). Briefly, digestion buffer and proteinase K were added to plasma samples, which were mixed and digested at 37° C. for 2 hours. Following digestion, binding buffer was added and samples were mixed. Isopropanol was added and samples were mixed. Sample lysates were passed through a silica-membrane column and washed with RNA Prep Buffer and RNA Wash Buffer. cfRNA was eluted in RNA Recovery Buffer and subsequently washed and concentrated in Zymo-Spin IC columns (Cat. No. C1004, Zymo Research Corp). Cleaned and concentrated cfRNA were eluted in 15 μl DNase/RNase-free water (Cat. No. W1001, Zymo Research Corp) then promptly stored at −20° C.

### 5.1.7 Method 7: Analysis of Purified cfRNA

Purified cfRNA from plasma samples were assessed by reverse transcription quantitative PCR (rt-qPCR) of two microRNAs: hsa-miR-16-5p (endogenous microRNA highly abundant in plasma for general cfRNA quantitative assessment) and hsa-miR-451a (microRNA highly enriched red blood cells and used specifically for hemolysis assessment) using the rt-qPCR protocol described in Cirera and Busk (Methods in Molecular Biology, 2014; 1182:73-81). In brief, 2 μl of cfRNA eluates were used to synthesize cfDNA in 10 μl reaction volume using poly(A) tail extension (Cat. No. M0276S, New England BioLabs) and M-MuLV reverse transcriptase (Cat. No. M0253S, New England BioLabs). The resulting cDNA was diluted 1:5 in nuclease-free water and 4 μl of the diluted cDNA was used for quantitative rt-qPCR in 10 μl reactions. The forward and reverse primer sequences for hsa-miR-16-5p were 5′ CGC AGT AGC AGC ACG TA 3′ (SEQ ID NO:3) and 5′ CAG TTT TTT TTT TTT TTT CGC CAA 3′(SEQ ID NO:4), respectively. The forward and reverse primer sequences for hsa-miR-451a were 5′ CGC AGA AAC CGT TAC CA 3′ (SEQ ID NO:5) and 5′ TTC AGT TTT TTT TTT TTT TTA ACT CAG T 3′(SEQ ID NO:6), respectively. The following cycle conditions were used for rt-qPCR: 95° C. for 5 min, 40 cycles of: 95° C. for 15 sec then 60° C. for 45 sec. Quantitative rt-qPCR reactions were run on CFX96 Touch™ Real-Time PCR Detection System (Model No. 1855195, Bio-Rad Laboratories, Inc.). The average of duplicates for each sample was used to obtain the cycle threshold (Ct) value.

### 5.1.8 Method 8: Analysis of Purified Blood Pellet gDNA

Following plasma separation as described in Method 1, the remaining pelleted blood cell fractions were used to obtain genomic DNA using Quick-DNA Mini Plus (Cat. No. D4068, Zymo Research). In brief, 100 μl of pelleted blood cell fraction was mixed with digestion buffer and proteinase K, incubated at 55° C. for 10 min, and mixed with binding buffer. The resulting lysate was passed through a silica-membrane column, which was washed with provided wash buffers, then eluted in 50 μl. Of the 50 μl total eluate, 10 μl was run on 1% agarose gel for 90 min at constant 90 V, then visualized using a UV light plate.

### 5.1.9 Method 9: Preparation of Aqueous Compositions

Stock solutions used herein are listed in Table 1. All stock solutions were prepared in water, with the exception of Q-VD-OPh.

TABLE 1
Components for preparation of aqueous compositions

| | | Component vol. | | | | Conc. post | |
|---|---|---|---|---|---|---|---|
| | | per 1 ml stock | | Conc. in | | blood | |
| Component | Stock conc. | solution | | Composition | | addition | |
| Polyvinylpyrrolidone, | 50% w/v in water | 300 | μl | 15% | w/v | 1.5% | w/v |
| 10,000 MW (PVP-10000) | | | | | | | |
| Polyethylene glycol | 50% w/v in water | 300 | μl | 15% | w/v | 1.5% | w/v |
| dimethyl ether, | | | | | | | |
| 2,000 MW (dmPEG-2000) | | | | | | | |
| Q-VD-OPh (pan- | 10 mM in Dimethyl | 50 | μl | 500 | μM | 50 | μM |
| caspase inhibitor) | sulfoxide (DMSO) | | | | | | |
| Saline concentrate | 2740 mM NaCl | 50 | μl | 137 mM NaCl | | 13.7 mM NaCl | |
| (NaCl and KCl) | 54 mM KCl | | | 2.7 mM KC1 | | 0.27 mM KCl | |
| K₂EDTA (anticoagulant) | 1 M | 100 | μl | 100 | mM | 10 | mM |
| Glucose | 4 M | 25 | μl | 100 | mM | 10 | mM |
| Urea | 1 M | 40 | μl | 40 | mM | 4 | mM |
| Naringin | 40 M Naringin | 1 | μl | 400 | nM | 40 | nM |
| Caffeine | 2 mM Caffeine | 1 | μl | 20 | μM | 2 | μM |

### 5.2 Example 1

This Example demonstrates that an aqueous composition comprising the polymer polyethylene glycol (PEG) and the pan-caspase apoptosis inhibitor, Q-VD-OPh, prevented apoptosis, stabilized plasma cell-free DNA (cfDNA), and prevented cfDNA dilution by cellular gDNA released by apoptotic cells.

To determine if inhibition of apoptosis can prevent cellular lysis, cellular DNA spillage and contamination of plasma, whole blood samples were incubated with various aqueous compositions, described below, in the presence or absence of PEG and the pan-caspase inhibitor, Q-VD-OPh.

Three aqueous compositions were prepared: saline solution (Composition 1) (137 mM NaCl, 2.7 mM KCl), saline solution with 20% w/v polyethylene glycol, 3350 MW (20% w/v PEG-3350) (Composition 2), and saline solution with 20% w/v PEG-3350 and 300 μM Q-VD-OPh VD-OPh (Composition 3), as described in Table 2 below (middle column). Whole blood was mixed with the aqueous compositions at a ratio of 9 parts blood to 1 part aqueous composition to produce the resulting mixtures described in Table 2 (right column). The resulting mixtures were incubated at ambient temperature (20-25° C.) for up to 4 days.

TABLE 2
Aqueous compositions comprising PEG-3350
in the presence or absence of Q-VD-OPh

| | Concentration of | Final concentration of components |
|---|---|---|
| Aqueous | components in | (after addition of blood to |
| Composition | aqueous composition | composition in ratio of 9:1) |
| Composition 1 | 137 mM NaCl; 2.7 mM KCl | 13.7 mM NaCl; 0.27 mM KCl |
| (“saline solution”) | | (Saline) |
| Composition 2 | 137 mM NaCl; 2.7 mM KCl; | 13.7 mM NaCl; 0.27 mM KCl; |
| | 20% w/v PEG-3350 | 2% w/v PEG-3350 (PEG-3350, 2%) |
| Composition 3 | 137 mM NaCl; 2.7 mM KCl; | 13.7 mM NaCl; 0.27 mM KCl; |
| | 20% w/v PEG-3350; | 2% w/v PEG-3350; |
| | 300 μM Q-VD-OPh | 30 μM Q-VD-OPh |
| | | (PEG-3350, 2%; Q-VD-OPh, 30 μM) |

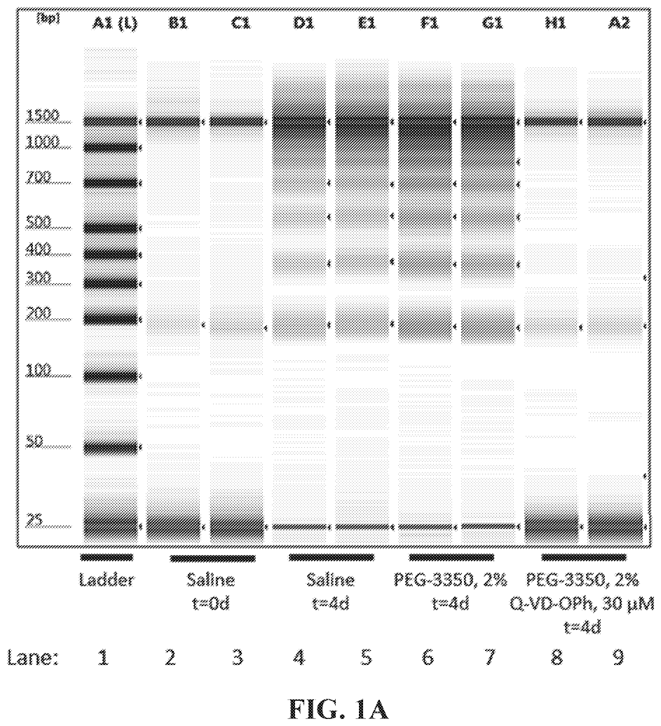

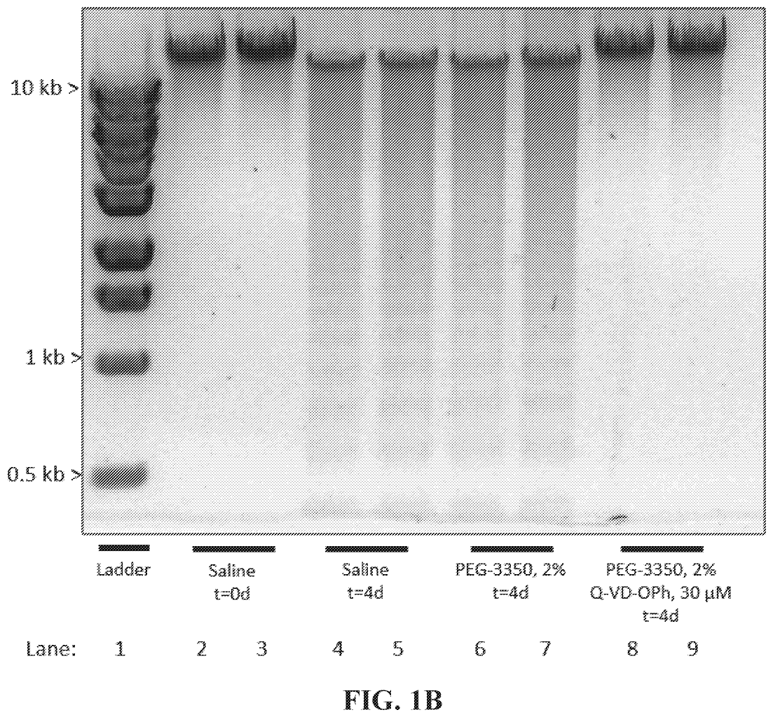

Blood samples were collected immediately, at day zero (t=0 d), or after a 4-day incubation at ambient temperature 4 (t=4 d), and plasma was separated as described in Method 1. Following plasma separation, cfDNA was isolated from plasma as described in Method 3. cfDNA was analyzed using microcapillary gel electrophoresis as described in Method 4 (FIG. 1A). Pelleted blood gDNA was assessed by gel electrophoresis as described in Method 8 (FIG. 1B).

### Microcapillary Gel Electrophoresis Analysis of DNA

Microcapillary gel electrophoresis of plasma collected immediately (t=0 d) from blood samples incubated with saline solution (Composition 1) resulted in a distinct band visible at 160 bp (FIG. 1A, lanes 2 and 3). Following incubation of blood with saline solution (Composition 1) for four days (t=4 d), an increase in the amount of plasma cfDNA was observed, migrating in a characteristic 160-180 bp apoptotic ladder pattern (FIG. 1A, lanes 4 and 5). This DNA fragmentation pattern was indicative of an apoptosis-induced release of fragmented gDNA into the extracellular environment, which can dilute cfDNA present.

Incubation of blood with an aqueous composition comprising PEG-3350 (Composition 2) resulted in an apoptotic ladder pattern at t=4 d similar to that observed with saline solution at t=4 d (Composition 1) (FIG. 1A, lanes 6 and 7, and 4 and 5, respectively). When blood was incubated with an aqueous composition comprising PEG-3350 and Q-VD-OPh (Composition 3), no apoptotic ladder cfDNA fragmentation pattern was observed (FIG. 1A, lanes 8 and 9). Rather, the cfDNA banding pattern was similar to that observed in the blood sample that was incubated with saline solution (Composition 1) and immediately processed (FIG. 1A, lanes 2 and 3). These results demonstrated that incubation of blood with a composition comprising PEG-3350 and the apoptosis inhibitor Q-VD-OPh inhibited genomic DNA (gDNA) degradation and release from cells, contaminating cfDNA.

### Analysis of Purified Blood Pellet gDNA

To determine if Q-VD-OPh had a similar effect on blood cell gDNA (as compared to gDNA released from the blood cells into the plasma fraction), gDNA was isolated from pelleted blood cell fractions of the samples represented in FIG. 1A. gDNA samples were prepared and analyzed using gel electrophoresis as described in Method 8. Each sample was prepared in duplicate.

A similar pattern of gDNA degradation was observed in FIG. 1B as that observed for the cfDNA in FIG. 1A. Blood cell pellets collected immediately from blood incubated with saline solution (Composition 1) showed little gDNA degradation (FIG. 1B, lanes 2 and 3). Following 4 days of incubation at ambient temperature, gDNA from samples incubated with saline solution (Composition 1) showed significant gDNA degradation with a fragmentation pattern typical of apoptosis (FIG. 1B, lane 4 and 5).

When blood was incubated with an aqueous composition comprising PEG-3350 (Composition 2) at t=4 d, an apoptotic ladder gDNA fragmentation pattern was observed (FIG. 1B, lanes 6 and 7). However, when blood was incubated with an aqueous composition comprising both PEG-3350 and Q-VD-OPh (Composition 3), no apoptotic ladder gDNA fragmentation pattern was observed at t=4 d (FIG. 1B, lanes 8 and 9). This gDNA fragmentation pattern is similar to the cfDNA fragmentation pattern observed from blood samples incubated with the same solution (Composition 3) (FIG. 1A, lanes 8 and 9), suggesting that the increase in fragmented DNA observed in plasma from blood samples incubated at ambient temperature for four days is largely due to the release of gDNA from apoptotic nucleated blood cells. The gDNA fragmentation pattern also suggests that incubation of blood with a composition comprising PEG-3350 and the apoptosis inhibitor Q-VD-OPh can prevent the apoptosis-induced release of fragment gDNA, diluting cfDNA present.

### 5.3 Example 2

This Example demonstrates that incubation of blood with an aqueous composition comprising PEG-3350 and Q-VD-OPh can simultaneously inhibit hemolysis and cellular apoptosis.

Polyethylene glycol (PEG) polymers of various polymer lengths and concentrations were tested for their ability, alone or in the presence of an apoptosis inhibitor, to inhibit hemolysis. PEG has been shown to reduce the lysis of blood cells due to mechanical stress (Kameneva M. V. et al., ASAIO J. (2003) 49(5):537-42). This is especially important for red blood cells, which are known to be prone to physical damage and are easily lysed compared to white blood cells.

The following aqueous compositions were prepared: saline solution (Composition 1) (137 mM NaCl, 2.7 mM KCl ); saline solution mixed with PEG 200, 1,000, 3,350, 6,000, 8,000, 10,000, and 20,000 weight average molecular weight (PEG-200, PEG-1000, PEG-3350, PEG-6000, PEG-8000, PEG-10000, and PEG-20000, respectively) at concentrations ranging from 2.5% to 30% w/v (Compositions 4 to 18); and saline solution mixed with PEG-3350 and Q-VD-OPh at concentrations of 100, 300, and 1000 μM (Compositions 19, 20, and 21, respectively). The concentrations of components in each aqueous composition are listed in Table 3 below (middle column). Whole blood was mixed with these aqueous compositions at a ratio of 9 parts blood to 1 part aqueous composition to produce the resulting mixtures described in Table 3 (right column). The resulting mixtures were incubated at ambient temperature (20-25° C.) for up to 3 days.

TABLE 3
Aqueous compositions comprising PEG polymers of various lengths
and concentrations in the presence or absence of Q-VD-OPh

| | Concentration of | Final concentration of components |
|---|---|---|
| Aqueous | components in | in resulting mixture following |
| Composition | aqueous composition | addition of blood at a ratio of 9:1 |
| Composition 1 | 137 mM NaCl; 2.7 mM KCl | 13.7 mM NaCl; 0.27 mM KCl |
| (“saline solution”) | | (Saline) |
| Composition 2 | 137 mM NaCl; 2.7 mM KCl; | 13.7 mM NaCl; 0.27 mM KCl; |
| | 20% w/v PEG-3350 | 2% w/v PEG-3350 (PEG-3350, 2%) |
| Composition 3 | 137 mM NaCl; 2.7 mM KCl; | 13.7 mM NaCl; 0.27 mM KCl; |
| | 20% w/v PEG-3500; | 2% w/v PEG-3500; |
| | 300 μM Q-VD-OPh | 30 μM Q-VD-OPh |
| | | (PEG-3500, 2% Q-VD-OPh, 30 μM) |
| Composition 4 | 137 mM NaCl; 2.7 mM KCl; | 13.7 mM NaCl; 0.27 mM KCl; |
| | 20% w/v PEG-200 | 2% w/v PEG-200 (PEG-200, 2%) |
| Composition 5 | 137 mM NaCl; 2.7 mM KCl; | 13.7 mM NaCl; 0.27 mM KCl; |
| | 30% w/v PEG-200 | 3% w/v PEG-200 (PEG-200, 3%) |
| Composition 6 | 137 mM NaCl; 2.7 mM KCl; | 13.7 mM NaCl; 0.27 mM KCl; |
| | 20% w/v PEG-1000 | 2% w/v PEG-1000 (PEG-1000, 2%) |
| Composition 7 | 137 mM NaCl; 2.7 mM KCl; | 13.7 mM NaCl; 0.27 mM KCl; |
| | 30% w/v PEG-1000 | 3% w/v PEG-1000 (PEG-1000, 3%) |
| Composition 8 | 137 mM NaCl; 2.7 mM KCl; | 13.7 mM NaCl; 0.27 mM KCl; |
| | 10% w/v PEG-3350 | 1% w/v PEG-3350 (PEG-3350, 1%) |
| Composition 9 | 137 mM NaCl; 2.7 mM KCl; | 13.7 mM NaCl; 0.27 mM KCl; |
| | 10% w/v PEG-6000 | 1% w/v PEG-6000 (PEG-6000, 1%) |
| Composition 10 | 137 mM NaCl; 2.7 mM KCl; | 13.7 mM NaCl; 0.27 mM KCl; |
| | 20% w/v PEG-6000 | 2% w/v PEG-6000 (PEG-6000, 2%) |
| Composition 11 | 137 mM NaCl; 2.7 mM KCl; | 13.7 mM NaCl; 0.27 mM KCl; |
| | 5% w/v PEG-8000 | 0.5% w/v PEG-8000 (PEG-8000, 0.5%) |
| Composition 12 | 137 mM NaCl; 2.7 mM KCl; | 13.7 mM NaCl; 0.27 mM KCl; |
| | 10% w/v PEG-8000 | 1% w/v PEG-8000 (PEG-8000, 1%) |
| Composition 13 | 137 mM NaCl; 2.7 mM KCl; | 13.7 mM NaCl; 0.27 mM KCl; |
| | 5% w/v PEG-10000 | 0.5% w/v PEG-10000 (PEG-10000, 0.5%) |
| Composition 14 | 137 mM NaCl; 2.7 mM KCl; | 13.7 mM NaCl; 0.27 mM KCl; |
| | 10% w/v PEG-10000 | 1% w/v PEG-10000 (PEG-10000, 1%) |
| Composition 15 | 137 mM NaCl; 2.7 mM KCl; | 13.7 mM NaCl; 0.27 mM KCl; |
| | 2.5% w/v PEG-20000 | 0.25% w/v PEG-20000 (PEG-20000, 0.25%) |
| Composition 16 | 137 mM NaCl; 2.7 mM KCl; | 13.7 mM NaCl; 0.27 mM KCl; |
| | 5% w/v PEG-20000 | 0.25% w/v PEG-20000 (PEG-20000, 0.5%) |
| Composition 17 | 137 mM NaCl; 2.7 mM KCl; | 13.7 mM NaCl; 0.27 mM KCl; |
| | 2.5% w/v PEG-3500 | 0.25% w/v PEGG500 (PEGG500, 0.25%) |
| Composition 18 | 137 mM NaCl; 2.7 mM KCl; | 13.7 mM NaCl; 0.27 mM KCl; |
| | 5% w/v PEG-3500 | 0.5% w/v PEGG 500 (PEG-3500, 0.5%) |
| Composition 19 | 137 mM NaCl; 2.7 mM KCl; | 13.7 mM NaCl; 0.27 mM KCl; |
| | 100 μM Q-VD-OPh | 10 μM Q-VD-OPh |
| Composition 20 | 137 mM NaCl; 2.7 mM KCl; | 13.7 mM NaCl; 0.27 mM KCl; |
| | 300 μM Q-VD-OPh | 30 μM Q-VD-OPh |
| Composition 21 | 137 mM NaCl; 2.7 mM KCl; | 13.7 mM NaCl; 0.27 mM KCl; |
| | 1000 μM Q-VD-OPh | 100 μM Q-VD-OPh |

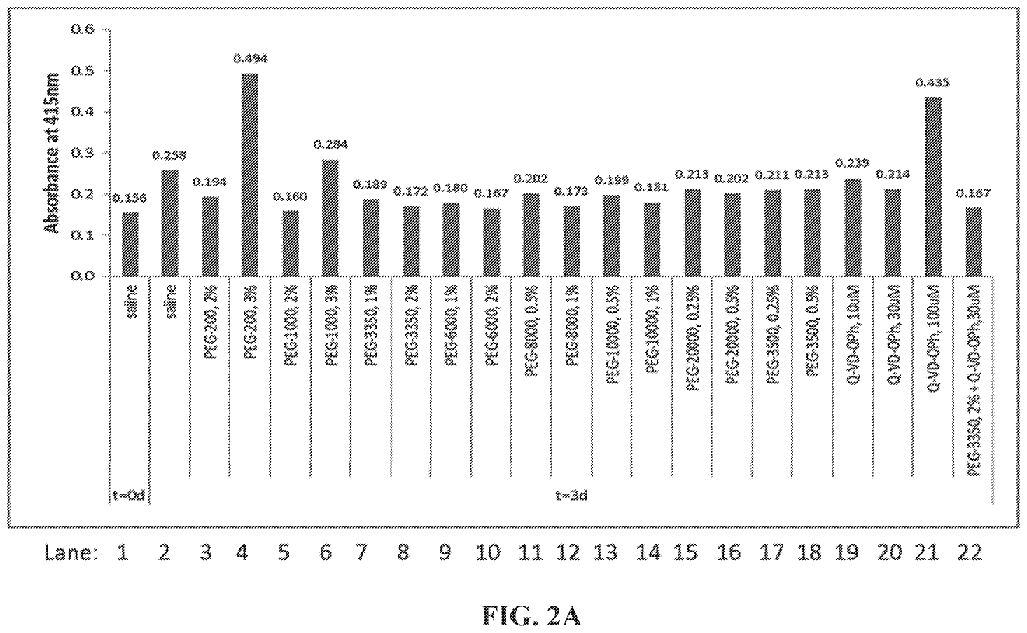

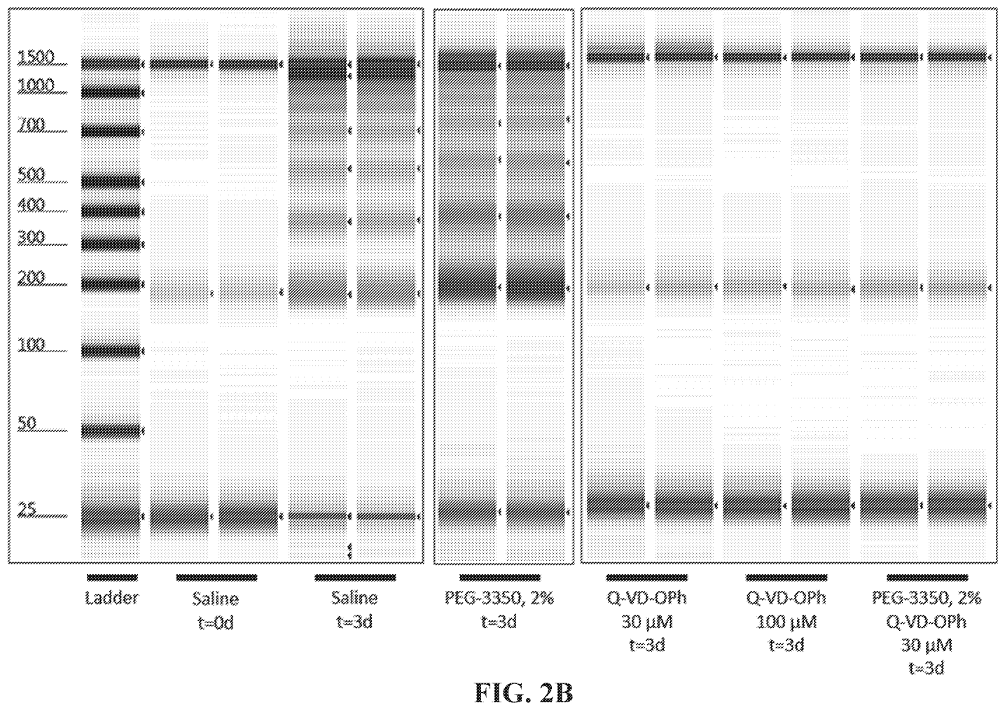

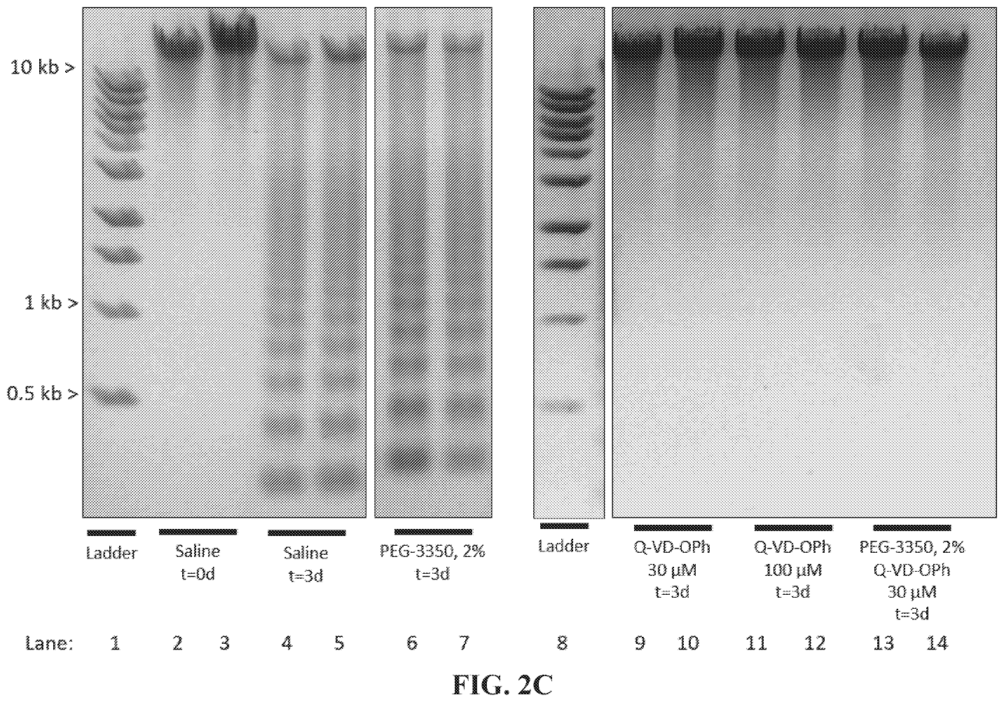

Blood samples were collected immediately (t=0 d) or after a 3-day incubation at ambient temperature (t=3 d) and plasma was separated described in Method 1. Hemolysis was assessed by UV-Vis spectrophotometry as described in Method 2 (FIG. 2A). Plasma cfDNA was isolated and assessed by microcapillary gel electrophoresis as described in Methods 3 and 4, respectively (FIG. 2B). Pelleted blood gDNA was assessed by gel electrophoresis as described in Method 8 (FIG. 2C).

### Analysis of Blood Hemolysis in Plasma

The amount of hemolysis was determined by measuring the absorbance of plasma samples at 415 nm (A₄₁₅) (FIG. 2A). Lane 1 shows the A₄₁₅ of plasma processed immediately after mixing with saline solution (Composition 1; t=0 d), and lanes 2-21 show the A₄₁₅ of plasma samples isolated from blood after a 3-day incubation with the indicated aqueous solution at ambient temperature (t=3 d). As may be seen from lane 1 (Composition 1), the plasma sample showed an A₄₁₅ increased from 0.156 to 0.258, indicating hemolysis (FIG. 2A, lanes 1 and 2). As may be seen from lanes 2-21 for the various PEG-containing compositions (Compositions 2-21), hemolysis was also observed, but at different amounts, indicating that different PEG-containing compositions confer different protective effects. For example, incubation of blood with a 30% w/v PEG-200 composition (Composition 4) resulted in an A₄₁₅ of 0.49, indicating a high amount of hemolysis (FIG. 2A, lane 5). On the other hand, incubation of blood with a 20% w/v PEG-3350 (Composition 2), resulted in an A₄₁₅ of 0.172 (FIG. 2A, lane 8), which indicating a low amount of hemolysis, and thus, a stronger inhibitory or protective effect.

The aqueous composition comprising 20% w/v PEG-3350 (Composition 2) was selected for further analysis to determine if compositions comprising both PEG-3350 and an apoptosis inhibitor, e.g., Q-VD-OPh, can inhibit apoptosis-induced dilution of cfDNA. A blood sample incubated with an aqueous composition comprising both PEG-3350 at 2% w/v and Q-VD-OPh at 30 μM (Composition 3) (FIG. 2A, lane 22) exhibited an A₄₁₂ of 0.167. This absorbance is lower than the A₄₁₂ of 0.172 of the sample incubated with PEG-3350 alone (Composition 2) (FIG. 2A, lane 8), indicating that PEG-3350 and Q-VD-OPh in combination contribute to inhibition of hemolysis. As a control, a plasma sample incubated in an aqueous composition lacking PEG but with a final concentration of 30 μM Q-VD-OPh (Composition 20) exhibited an A₄₁₂=0.214 (FIG. 2A, lane 20), which value is greater than the A₄₁₂ of 0.156 of the immediately separated plasma (FIG. 2A, lane 1), but is similar to the A₄₁₂ of 0.258 of the saline solution (Composition 1) (FIG. 2A, lane 2).

### Microcapillary Gel Electrophoresis Analysis of DNA

To qualitatively assess the effect of aqueous compositions comprising PEG-3350 and Q-VD-OPh on the degradation of cfDNA, plasma cfDNA was isolated from samples described above and assessed by microcapillary gel electrophoresis as described in Method 4 (FIG. 2B). cfDNA isolated from blood samples incubated with 20% w/v PEG-3350 alone (Composition 2) resulted in an apoptotic ladder DNA fragmentation pattern that was similar to samples incubated with saline solution (Composition 1) (FIG. 2B, lanes 4-7). These results indicated that PEG-3350 alone cannot inhibit formation of the apoptotic ladder pattern that resulted from contamination of cfDNA with gDNA (FIG. 2A, lanes 6 and 7). However, cfDNA isolated from blood samples incubated with an aqueous composition of 30 μM Q-VD-OPh (Composition 20) or 100 μM Q-VD-OPh (Composition 21) resulted in inhibition of apoptosis and a distinct banding pattern (FIG. 2B, lanes 8-11). [This banding pattern is similar to that observed in the cfDNA isolated from plasma immediately collected from a saline solution (Composition 1) (FIG. 2B, lanes 2 and 3). The cfDNA isolated from blood incubated with an aqueous composition of PEG-3350 and 300 μM Q-VD-OPh (Composition 3) resulted in a similar pattern, indicating that this composition can prevent gDNA spillage into the blood plasma (FIG. 2B, lanes 12 and 13).

Taken together, these results indicate that incubation of blood with certain compositions containing PEG-3350 and Q-VD-OPh can minimize hemolysis and inhibit apoptosis.

### Analysis of Purified Blood Pellet gDNA

To confirm the finding that incubation of blood samples with an aqueous composition comprising 20% w/v PEG-3350, and 300 μM Q-VD-OPh (Composition 3) can inhibit apoptosis, gDNA was analyzed for signs of fragmentation. Following plasma separation, gDNA was isolated from blood pellets and analyzed by gel electrophoresis as described in Method 8. Blood samples incubated in saline solution (Composition 1) or 20% w/v PEG-3350 (Composition 2) exhibited a DNA fragmentation pattern indicative of apoptosis (FIG. 2C, lanes 4-7). These results confirm that incubation of blood samples with an aqueous composition of 20% w/v PEG-3350 and 300 μM Q-VD-OPh (Composition 3) does not exhibit the DNA fragmentation pattern indicative of apoptosis (FIG. 2C, lanes 13 and 14).

Taken together, these results demonstrate that incubation of blood with an aqueous composition comprising PEG-3350 and Q-VD-OPh can simultaneously inhibit hemolysis and inhibit cellular apoptosis.

### 5.4 Example 3

This Example demonstrates that an aqueous composition comprising polyvinylpyrrolidone (PVP) and apoptosis inhibitor showed significant inhibition of hemolysis in blood samples.

To assess the ability of different polymers to inhibit hemolysis in blood samples, polypropylene glycol (PPG), polyvinylpyrrolidone (PVP), and polyethylene glycol dimethyl ether (dmPEG) polymers of various molecular weights and concentrations were tested and compared to PEG-3350.

The following aqueous compositions were prepared: saline solution mixed with PPG, PVP, and dmPEG polymers of varying weight average molecular weights ranging from 425 to 40,000, and at varying concentrations ranging from 5% to 40% w/v (Compositions 22-32). These compositions were compared to an aqueous composition comprising PEG-3350 (Composition 3). Except for the saline solution (Composition 1), all compositions contained 500 μM Q-VD-OPh.

The concentrations of components in each aqueous composition are listed in Table 4 below (middle column). Whole blood was mixed with these aqueous compositions at a ratio of 9 parts blood to 1 part aqueous composition to produce the resulting mixtures described in Table 4 (right column). The resulting mixtures were incubated at ambient temperature (20-25° C.) for up to 5 days.

TABLE 4
Aqueous compositions comprising Q-VD-OPh and polymers
of various lengths and concentrations

| | Concentration of | Final concentration of components |
|---|---|---|
| Aqueous | components in | (after addition of blood to |
| Composition | aqueous composition | composition in ratio of 9:1) |
| Composition 1 | 137 mM NaCl; 2.7 mM KCl | 13.7 mM NaCl; 0.27 mM KCl |
| (“saline solution”) | | (Saline) |
| Composition 3 | 137 mM NaCl; 2.7 mM KCl; | 13.7 mM NaCl; 0.27 mM KCl; |
| | 20% w/v PEG-3350; | 2% w/v PEG-3350; |
| | 500 μM Q-VD-OPh | 50 μM Q-VD-OPh (PEG-3350, 2%) |
| Composition 22 | 137 mM NaCl; 2.7 mM KCl; | 13.7 mM NaCl; 0.27 mM KCl; |
| | 10% w/v PPG-425; | 1% w/v PPG-425; |
| | 500 μM Q-VD-OPh | 50 μM Q-VD-OPh (425, 1%) |
| Composition 23 | 137 mM NaCl; 2.7 mM KCl; | 13.7 mM NaCl; 0.27 mM KCl; |
| | 20% w/v PPG-425; | 2% w/v PPG-425; |
| | 500 μM Q-VD-OPh | 50 μM Q-VD-OPh (425 2%) |
| Composition 24 | 137 mM NaCl; 2.7 mM KCl; | 13.7 mM NaCl; 0.27 mM KCl; |
| | 5% w/v PPG-1000; | 0.5% w/v PPG-1000; |
| | 500 μM Q-VD-OPh | 50 μM Q-VD-OPh (1000 0.5%) |
| Composition 25 | 137 mM NaCl; 2.7 mM KCl; | 13.7 mM NaCl; 0.27 mM KCl; |
| | 10% w/v PPG-1000; | 1% w/v PPG-1000; |
| | 500 μM Q-VD-OPh | 50 μM Q-VD-OPh (1000 1%) |
| Composition 26 | 137 mM NaCl; 2.7 mM KCl; | 13.7 mM NaCl; 0.27 mM KCl; |
| | 15% w/v PVP-10000; | 1.5% w/v PPG-10000; |
| | 500 μM Q-VD-OPh | 50 μM Q-VD-OPh (10k 1.5%) |
| Composition 27 | 137 mM NaCl; 2.7 mM KCl; | 13.7 mM NaCl; 0.27 mM KCl; |
| | 10% w/v PVP-40,000; | 1% w/v PPG-40,000; |
| | 500 μM Q-VD-OPh | 50 μM Q-VD-OPh (40k 1%) |
| Composition 28 | 137 mM NaCl; 2.7 mM KCl; | 13.7 mM NaCl; 0.27 mM KCl; |
| | 20% w/v dmPEG-500; | 2% w/v dmPEG-500; |
| | 500 μM Q-VD-OPh | 50 μM Q-VD-OPh (500 2%) |
| Composition 29 | 137 mM NaCl; 2.7 mM KCl; | 13.7 mM NaCl; 0.27 mM KCl; |
| | 40% w/v dmPEG-500; | 4% w/v dmPEG-500; |
| | 500 μM Q-VD-OPh | 50 μM Q-VD-OPh (500 4%) |
| Composition 30 | 137 mM NaCl; 2.7 mM KCl; | 13.7 mM NaCl; 0.27 mM KCl; |
| | 20% w/v dmPEG-1000; | 2% w/v dmPEG-1000; |
| | 500 μM Q-VD-OPh | 50 μM Q-VD-OPh (1000 2%) |
| Composition 31 | 137 mM NaCl; 2.7 mM KCl; | 13.7 mM NaCl; 0.27 mM KCl; |
| | 10% w/v dmPEG-2000; | 1% w/v dmPEG-2000; |
| | 500 μM Q-VD-OPh | 50 μM Q-VD-OPh (2000 1%) |
| Composition 32 | 137 mM NaCl; 2.7 mM KCl; | 13.7 mM NaCl; 0.27 mM KCl; |
| | 20% w/v dmPEG-2000; | 2% w/v dmPEG-2000; |
| | 500 μM Q-VD-OPh | 50 μM Q-VD-OPh (2000 2%) |

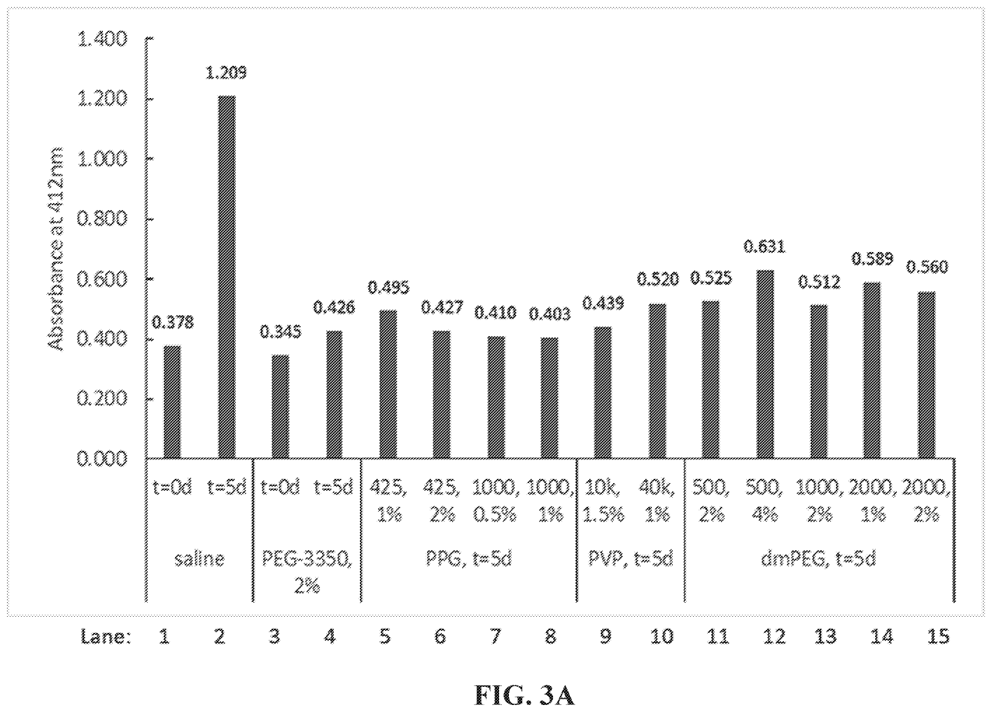

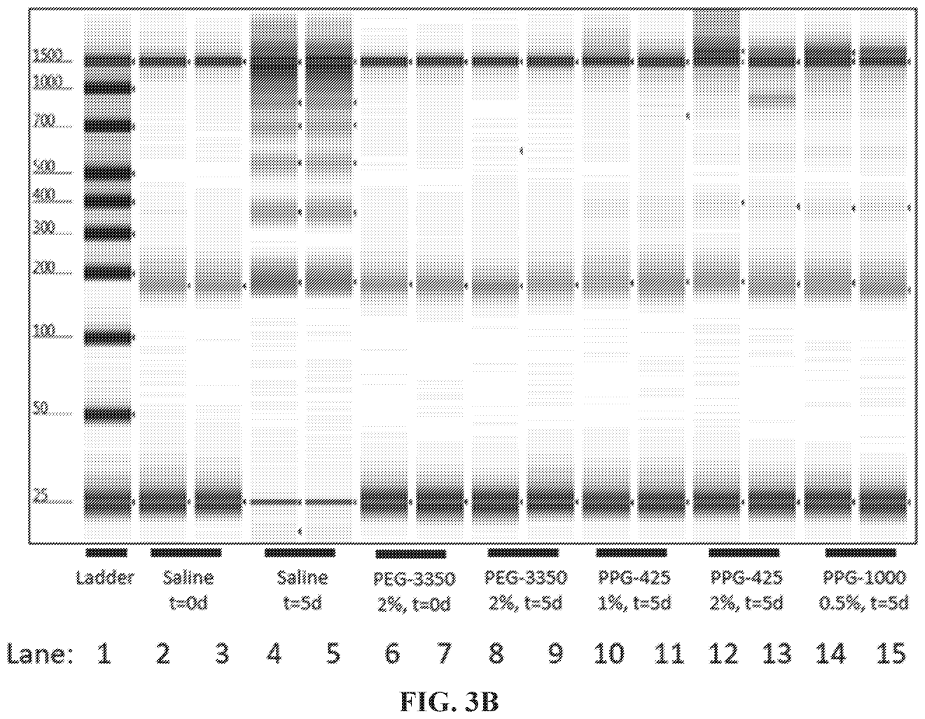

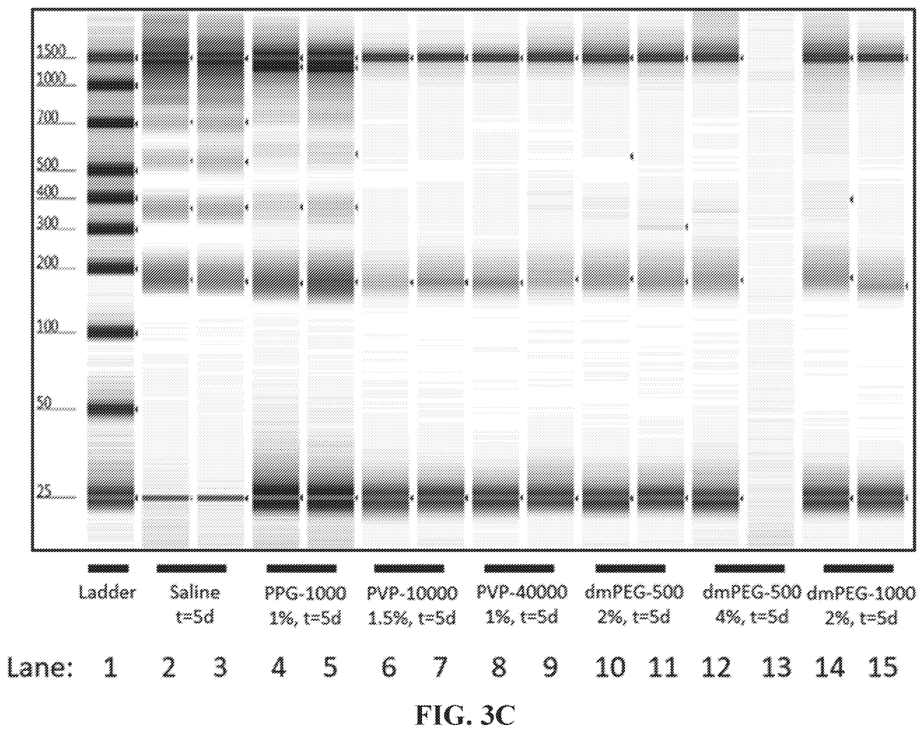

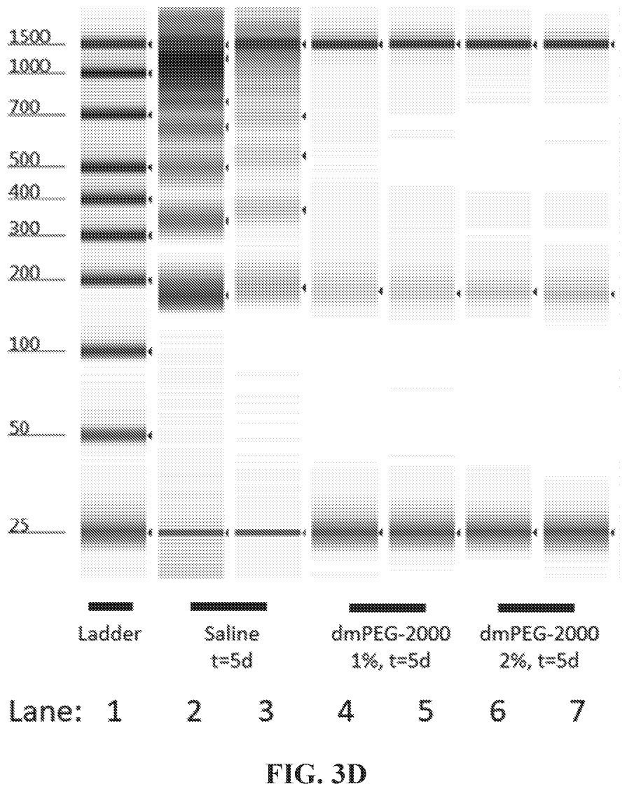

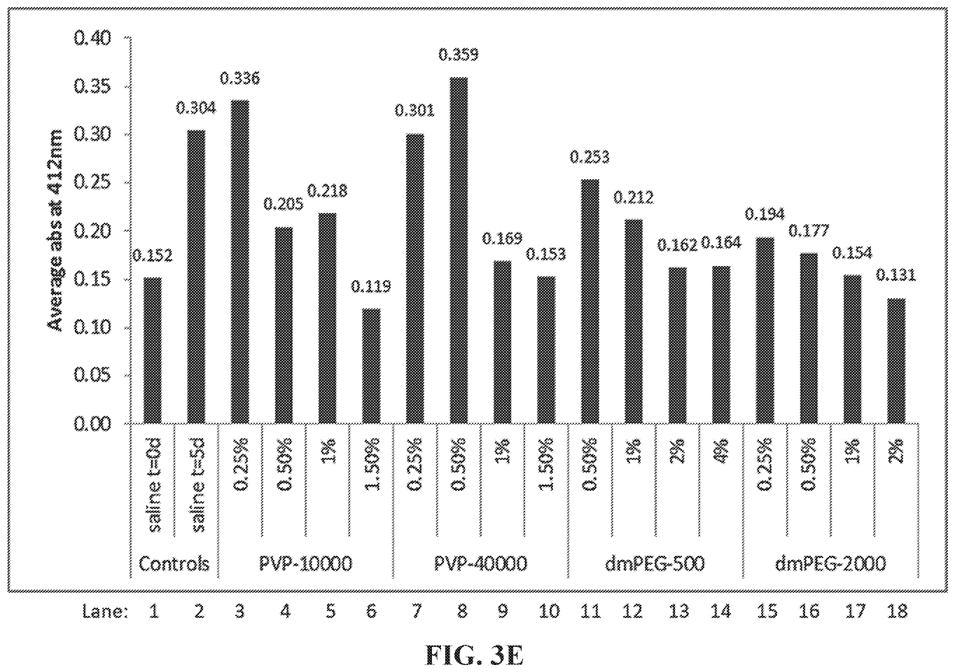

Plasma was separated immediately (t=0 d) or after a 5-day incubation at ambient temperature (t=5 d) as described in Method 1. Plasma hemolysis was assessed by UV-Vis spectrophotometry as described in Method 2 (FIG. 3A and 3E), and plasma cfDNA was isolated and assessed by microcapillary gel electrophoresis as described in Methods 3 and 4, respectively (FIG. 3B-3D).

### Analysis of Blood Hemolysis in Plasma

Blood samples incubated with aqueous compositions comprising PVP, PPG and dmPEG polymers were assessed for hemolysis to determine if these compositions could improve the inhibition of hemolysis observed for 20% w/v PEG-3350 (compare, e.g., A₄₁₂=0.43 for Composition 3, FIG. 3A, lane 4, to A₄₁₂=1.2 for Composition 1, FIG. 3A, lane 2).

All tested aqueous compositions (Compositions 22-32) exhibited inhibition of hemolysis compared to saline solution (Composition 1) (FIG. 3A, lanes 4-15). Several polymers were as effective as, or more effective than, 20% w/v PEG-3350 (Composition 3) at inhibition of hemolysis. For example, incubation of blood with an aqueous composition of 10% w/v PPG-1000 (Composition 25) exhibited an A₄₁₂ of 0.40, and incubation with an aqueous composition of 15% w/v PVP-10000 (Composition 26) exhibited an A₄₁₂ of 0.44 (FIG. 3A, lanes 8 and 9, respectively). As may be seen from these data, compositions comprising PEG, PVP, PPG, and dmPEG polymers can effectively inhibit hemolysis in blood incubated at ambient temperature, and certain compositions comprising such polymers are more effective than others.

### Microcapillary Gel Electrophoresis Analysis of DNA and Analysis of Purified Blood Pellet gDNA

To investigate whether Q-VD-OPh can maintain its apoptosis inhibition activity in aqueous compositions comprising tested polymers, cfDNA and gDNA were isolated from samples and analyzed. Microcapillary gel electrophoresis of cfDNA isolated from plasma derived from blood samples incubated with PPG-425 (Compositions 22 and 23) or PPG-1000 (Compositions 24 and 25) for five days revealed an apoptotic ladder pattern, suggesting that PPG had negatively impacted the anti-apoptotic activity of Q-VD-OPh (FIG. 3B, lanes 10-15 and FIG. 3C, lanes 4-5). These results indicate that although compositions comprising PPG can inhibit hemolysis, certain PPG polymers are not compatible with the apoptosis inhibitor Q-VD-OPh.

In contrast, cfDNA from blood incubated with aqueous compositions comprising Q-VD-OPh and PVP (Compositions 26 and 27) exhibited no apoptosis ladder pattern, indicating that the apoptosis inhibitor was active in this composition (FIG. 3C, lanes 6-9). Similarly, cfDNA isolated from blood incubated with aqueous compositions comprising Q-VD-OPh and dmPEG (Compositions 28-32) demonstrated an inhibition of apoptosis (FIG. 3C, lanes 10-12, 14-15 (loading error in lane 13); and FIG. 3D, lanes 4-7). These results indicate that aqueous compositions comprising apoptosis inhibitor and either PVP or dmPEG polymers can significantly inhibit hemolysis and apoptosis of blood samples.

To further assess the ability of compositions comprising Q-VD-OPh and the polymers PVP and dmPEG to inhibit hemolysis, a wider range of polymer concentrations was analyzed. The concentrations of components in each aqueous composition are listed in Table 5 below (middle column). Whole blood was mixed with the aqueous compositions at a ratio of 9 parts blood to 1 part aqueous composition to produce the resulting mixtures described in Table 5 (right column). The resulting mixtures were incubated at ambient temperature (20-25° C.) for up to 5 days.

TABLE 5
Aqueous compositions comprising Q-VD-OPh and PVP and
dmPEG polymers of various lengths and concentrations

| | Concentration of | Final concentration of components |
|---|---|---|
| Aqueous | components in | (after addition of blood to |
| Composition | aqueous composition | composition in ratio of 9:1) |
| Composition 1 | 137 mM NaCl; 2.7 mM KCl | 13.7 mM NaCl; 0.27 mM KCl |
| (“saline solution”) | | (Saline) |
| Composition 26 | 137 mM NaCl; 2.7 mM KCl; | 13.7 mM NaCl; 0.27 mM KCl; |
| | 15% w/v PVP-10000; | 1.5% w/v PVP-10000; |
| | 500 μM Q-VD-OPh | 50 μM Q-VD-OPh (PVP-10000 1.5%) |
| Composition 33 | 137 mM NaCl; 2.7 mM KCl; | 13.7 mM NaCl; 0.27 mM KCl; |
| | 2.5% w/v PVP-10000; | 0.25% w/v PVP-10000; |
| | 500 μM Q-VD-OPh | 50 μM Q-VD-OPh (PVP-10000 0.25%) |
| Composition 34 | 137 mM NaCl; 2.7 mM KCl; | 13.7 mM NaCl; 0.27 mM KCl; |
| | 5% w/v PVP-10000; | 0.5% w/v PVP-10000; |
| | 500 μM Q-VD-OPh | 50 μM Q-VD-OPh (PVP-10000 0.5%) |
| Composition 35 | 137 mM NaCl; 2.7 mM KCl; | 13.7 mM NaCl; 0.27 mM KCl; |
| | 10% w/v PVP-10000; | 1% w/v PVP-10000; |
| | 500 μM Q-VD-OPh | 50 μM Q-VD-OPh (PVP-10000 1%) |
| Composition 27 | 137 mM NaCl; 2.7 mM KCl; | 13.7 mM NaCl; 0.27 mM KCl; |
| | 10% w/v PVP-40,000; | 1% w/vPVP-40,000; |
| | 500 μM Q-VD-OPh | 50 μM Q-VD-OPh (PVP-40000 1%) |
| Composition 36 | 137 mM NaCl; 2.7 mM KCl; | 13.7 mM NaCl; 0.27 mM KCl; |
| | 2.5% w/v PVP-40,000; | 0.25% w/v PVP-40,000; |
| | 500 μM Q-VD-OPh | 50 μM Q-VD-OPh (PVP-40000 0.25%) |
| Composition 37 | 137 mM NaCl; 2.7 mM KCl; | 13.7 mM NaCl; 0.27 mM KCl; |
| | 5% w/v PVP-40,000; | 0.5% w/v PVP-40,000; |
| | 500 μM Q-VD-OPh | 50 μM Q-VD-OPh (PVP-40000 0.5%) |
| Composition 38 | 137 mM NaCl; 2.7 mM KCl; | 13.7 mM NaCl; 0.27 mM KCl; |
| | 15% w/v PVP-40,000; | 1.5% w/v PVP-40,000; |
| | 500 μM Q-VD-OPh | 50 μM Q-VD-OPh (PVP-40000 1.5%) |
| Composition 28 | 137 mM NaCl; 2.7 mM KCl; | 13.7 mM NaCl; 0.27 mM KCl; |
| | 20% w/v dmPEG-500; | 2% w/v dmPEG-500; |
| | 500 μM Q-VD-OPh | 50 μM Q-VD-OPh (dmPEG-500 2%) |
| Composition 29 | 137 mM NaCl; 2.7 mM KCl; | 13.7 mM NaCl; 0.27 mM KCl; |
| | 40% w/v dmPEG-500; | 4% w/v dmPEG-500; |
| | 500 μM Q-VD-OPh | 50 μM Q-VD-OPh (dmPEG-500 4%) |
| Composition 39 | 137 mM NaCl; 2.7 mM KCl; | 13.7 mM NaCl; 0.27 mM KCl; |
| | 5% w/v dmPEG-500; | 0.5% w/v dmPEG-500; |
| | 500 μM Q-VD-OPh | 50 μM Q-VD-OPh (dmPEG-500 0.5%) |
| Composition 40 | 137 mM NaCl; 2.7 mM KCl; | 13.7 mM NaCl; 0.27 mM KCl; |
| | 10% w/v dmPEG-500; | 1% w/v dmPEG-500; |
| | 500 μM Q-VD-OPh | 50 μM Q-VD-OPh (dmPEG-500 1%) |
| Composition 31 | 137 mM NaCl; 2.7 mM KCl; | 13.7 mM NaCl; 0.27 mM KCl; |
| | 10% w/v dmPEG-2000; | 1% w/v dmPEG-2000; |
| | 500 μM Q-VD-OPh | 50 μM Q-VD-OPh (dmPEG-2000 1%) |
| Composition 32 | 137 mM NaCl; 2.7 mM KCl; | 13.7 mM NaCl; 0.27 mM KCl; |
| | 20% w/v dmPEG-2000; | 2% w/v dmPEG-2000; |
| | 500 μM Q-VD-OPh | 50 μM Q-VD-OPh (dmPEG-2000 2%) |
| Composition 41 | 137 mM NaCl; 2.7 mM KCl; | 13.7 mM NaCl; 0.27 mM KCl; |
| | 2.5% w/v dmPEG-2000; | 0.25% w/v dmPEG-2000; |
| | 500 μM Q-VD-OPh | 50 μM Q-VD-OPh (dmPEG-2000 0.25%) |
| Composition 42 | 137 mM NaCl; 2.7 mM KCl; | 13.7 mM NaCl; 0.27 mM KCl; |
| | 5% w/v dmPEG-2000; | 0.5% w/v dmPEG-2000; |
| | 500 μM Q-VD-OPh | 50 μM Q-VD-OPh (dmPEG-2000 0.5%) |

### Analysis of Blood Hemolysis in Plasma

The blood samples incubated with the aqueous composition listed in Table 5 were assessed for hemolysis. Plasma absorbance was compared to plasma from blood incubated with saline solution (Composition 1) for five days, which had an A₄₁₂ of 0.315 (FIG. 3E, lane 2).

Most tested compositions comprising PVP or dmPEG inhibited hemolysis, with the notable exception of compositions comprising 2.5% w/v PVP-10000 (Composition 33) and 5% w/v PVP-40,000 (Composition 37) (FIG. 3E, lanes 3 and 8, respectively).

Surprisingly, a five-day incubation of blood with an aqueous composition comprising 15% w/v PVP-10000 and 500 μM Q-VD-OPh (Composition 26) resulted in an A₄₁₂ of 0.119, demonstrating less hemolysis than that observed in the blood sample immediately separated from saline solution (Composition 1) (FIG. 3E, lanes 6 and 1, respectively). Thus, PVP-10000 significantly reduced hemolysis resulting from physical cellular stress (e.g., during the centrifugation step to separate plasma).

As a control, the effect of polymer concentration on absorbance was examined to determine whether polymers could be binding to free hemoglobin, thereby reducing the amount detected in the assay, leading to a concomitant reduction in A₄₁₂. Incubation of blood samples with aqueous compositions comprising 5% and 10% w/v PVP-40,000 (Composition 37 and 27, respectively) resulted in a decrease in A₄₁₂ from 0.359 to 0.169 (FIG. 3E, lanes 8 and 9, respectively). However, an increase in polymer concentration from 2.5% to 5% w/v PVP-40,000 (Compositions 36 and 37, respectively) resulted in an increase in A₄₁₂ from 0.301 to 0.359 (FIG. 3E, lanes 7 and 8, respectively). Therefore, although it is possible that the polymers bind free hemoglobin, the absence of a correlation between absorbance values and polymer concentration suggests that inhibition of hemolysis is the principal factor in the reduction in A₄₁₂ following incubation of blood with these PVP and dmPEG aqueous compositions.

In conclusion, an aqueous composition comprising 15% w/v PVP-10000 (Composition 26) reduced hemolysis and protected cfDNA from fragmentation and contamination by cellular DNA.

### 5.5 Example 4

This Example demonstrates that an aqueous composition comprising PVP, an apoptosis inhibitor, an anticoagulant, and one or more sugars can inhibit hemolysis for up to 7 days and stabilize cfDNA for up to 10 days.

The effect of long-term incubation of blood samples in an aqueous composition at ambient temperature was assessed up to, and beyond 5 days to assess the duration of effective preservation and stabilization. To aid in stabilization, sugars (glucose and trehalose) and the anticoagulant K₂EDTA were added to aqueous compositions tested. Without being bound to any particular theory, EDTA may chelate divalent metal ions, such as calcium and magnesium, which are required for enzymatic reactions, including the reactions necessary for the coagulation cascade.

The following composition was prepared: 137 mM NaCl; 2.7 mM KCl; 15% w/v PVP-10000; 100 mM K₂EDTA; 500 μM Q-VD-OPh; 10 mM D+Glucose; and 10 mM trehalose (Composition 43). Saline solution (Composition 1) (137 mM NaCl and 2.7 mM KCl) was used a control. The concentrations of components in the aqueous composition are listed in Table 6 below (middle column). Whole blood was mixed with the aqueous compositions at a ratio of 9 parts blood to 1 part aqueous composition to produce the resulting mixture described in Table 6 (right column). The resulting mixtures were incubated at ambient temperature (20-25° C.) for up to 14 days.

TABLE 6
An aqueous composition comprising PVP-10000,
apoptosis inhibitor, anticoagulant, and sugar

| | Concentration of | Final concentration of components |
|---|---|---|
| Aqueous | components in | (after addition of blood to |
| Composition | aqueous composition | composition in ratio of 9:1) |
| Solution 1 | 137 mM NaCl; 2.7 mM KCl | 13.7 mM NaCl; 0.27 mM KCl |
| (“saline solution”) | | (Saline) |
| Composition 43 | 137 mM NaCl; 2.7 mM KCl; | 13.7 mM NaCl; 0.27 mM KCl; |
| | 15% w/v PVP-10000; | 1.5% w/v PVP-10000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose | 1 mM trehalose (“preserve”) |

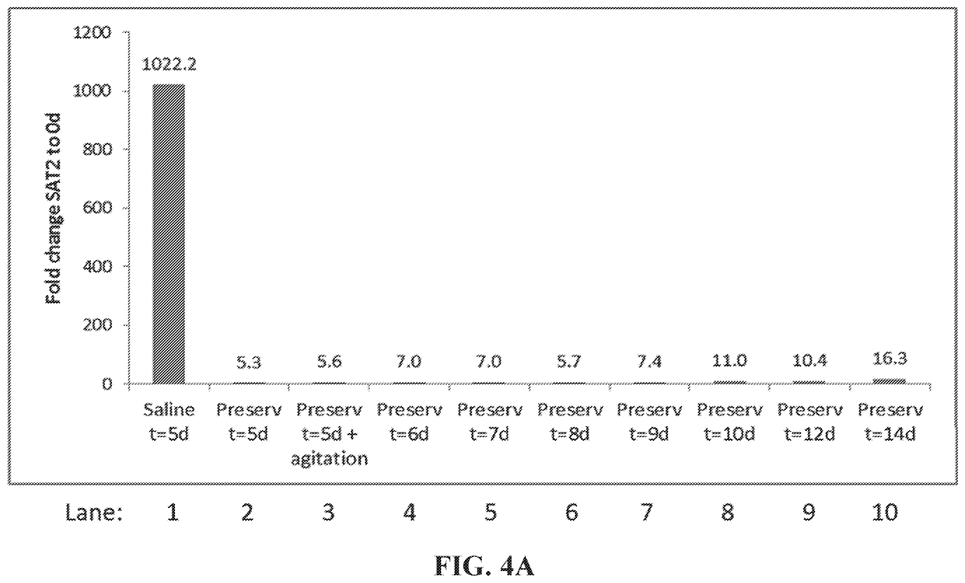

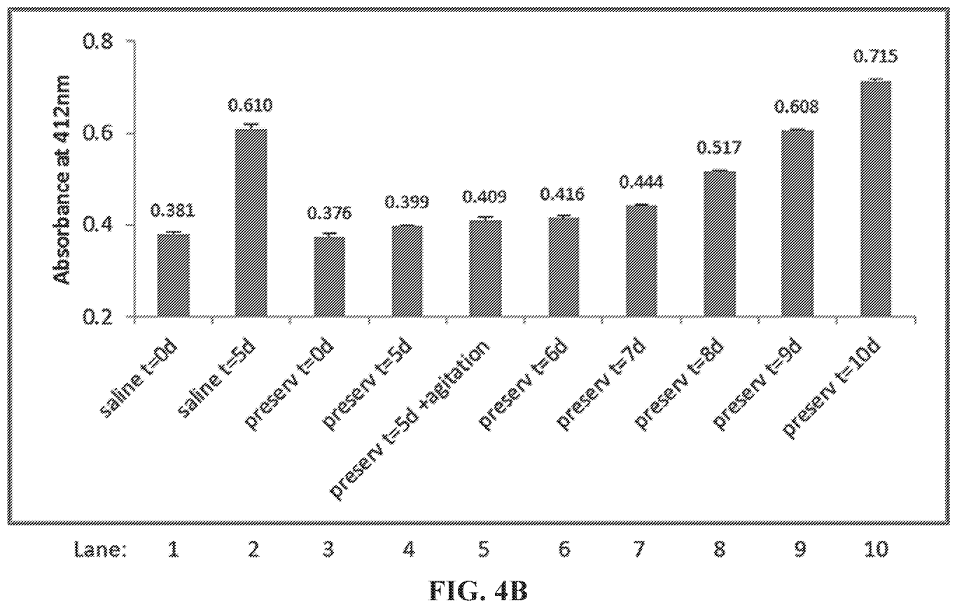

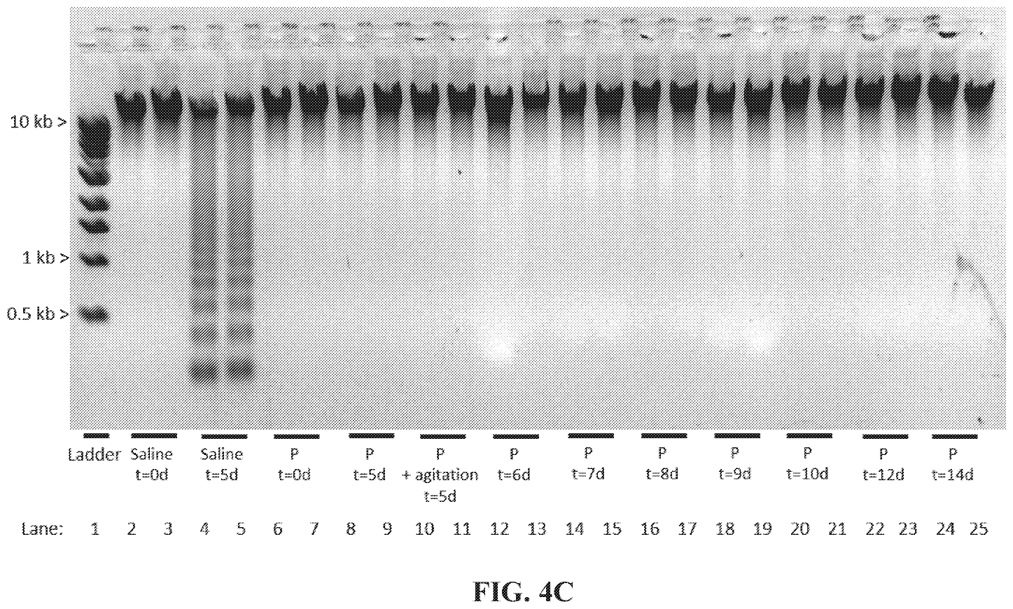

Blood samples were collected immediately (t=0 d) or after 5 days or more, up to 14 days, of incubation at ambient temperature (t=5 d to t=14 d). To simulate increased physical agitation due to harsh transport conditions, one blood sample was incubated at a ratio of 9 to 1 with the same aqueous composition (Composition 43) for five days, and ten 180° inversions were applied to the sample per incubation day (FIG. 4A, lane 3; FIG. 4B, lane 5; and FIG. 4C, lanes 10 and 11). Plasma was separated from samples as described in Method 1. Plasma cfDNA was isolated as described in Method 3, and quantification of plasma cfDNA was performed using PCR targeting the Sat2 locus fragment as described in Method 5 (FIG. 4A). Hemolysis was assessed by UV-Vis spectrophotometry as described in Method 2 (FIG. 4B). Pelleted blood gDNA was assessed by gel electrophoresis as described in Method 8 (FIG. 4C).

### Quantitative PCR Analysis of DNA

Plasma cfDNA contamination by gDNA spillage was quantified using quantitative PCR targeting the Sat2 locus as described in Method 5, and cycle threshold (Ct) values were determined. The Ct indicates how many cycles are required to detect a real signal from the sample and is inversely correlated to the amount of target nucleic acid in the sample.

It is generally believed that the Sat2 locus fragment remains tightly bound to histones, which protects the bound DNA from degradation in blood once released from dead cells. Accordingly, qPCR detection of Sat2 DNA fragment indicates plasma cfDNA contamination by gDNA. Results are depicted as the fold-change in the amount of Sat2 detected between the indicated day and t=0 (FIG. 4A).

The qPCR analysis of plasma collected from blood incubated with saline solution ((Composition 1) at t=5 d) indicated the presence of 1,022-fold more Sat2 fragment in plasma cfDNA compared to the blood sample collected at t=0 d (FIG. 4A, lane 1). However, incubation of blood in the aqueous composition (Composition 43) described in Table 6 resulted in the detection of only a 5.3-fold increase in Sat2 PCR product at t=5 and a 16.3-fold increase in Sat2 PCR product at t=14 (FIG. 4A, lanes 5 and 10, respectively). These results demonstrate that the aqueous composition comprising PVP, an apoptosis inhibitor, an anticoagulant, and one or more sugars, for example, the aqueous composition comprising 137 mM NaCl; 2.7 mM KCl; 15% w/v PVP-10000; 100 mM K₂EDTA; 500 μM Q-VD-OPh; 10 mM D+Glucose; and 10 mM trehalose (Composition 43), can significantly inhibit contamination of cfDNA in blood samples incubated at ambient temperature for at least 14 days. Additional physical agitation of blood incubated with aqueous composition (Composition 43) for 5 days only slightly increased the fold-change in the amount of Sat2 PCR product to 5.6 fold (FIG. 4A, lane 6), indicating effective cfDNA stabilization during a simulated harsh transport condition.

### Analysis of Blood Hemolysis in Plasma

Blood samples incubated with the aqueous composition comprising PVP, an apoptosis inhibitor, an anticoagulant, and one or more sugars (Composition 43) were also assessed for hemolysis. Plasma collected from blood sample incubated with saline solution (Composition 1) resulted in an increase in A₄₁₂ from 0.381 at t=0 to 0.610 at t=5 (FIG. 4B, lanes 1 and 2, respectively). However, plasma collected from blood incubated with the aqueous composition (Composition 43) inhibited the increase in A₄₁₂ to 0.399 after 5 days of incubation at ambient temperature (FIG. 4B, lane 4).

Additional physical agitation for 5 days at ambient temperature incubation period only slightly increased the absorbance to 0.409 (FIG. 4B, lane 5), demonstrating effective hemolysis inhibition in simulated harsh transport conditions.

### Analysis of Purified Blood Pellet gDNA

To assess the effect on gDNA, pelleted blood gDNA was isolated and visualized by gel electrophoresis as described in Method 8. (FIG. 4C). Blood samples that were incubated with the aqueous composition (Composition 43) showed highly effective inhibition of apoptosis, with no visible apoptotic ladder pattern of gDNA degradation from between 5 to 14 days of incubation (FIG. 4C, lanes 8 to 25). In contrast, when blood was incubated with saline solution (Composition 1) for five days, a clear apoptotic ladder pattern of gDNA degradation was observed (FIG. 4C, lanes 4 and 5). This result provides further evidence of a positive correlation between the amount of Sat2 in plasma cfDNA fractions detected by PCR and the degree of gDNA fragmentation.

Taken together, these results demonstrate that the aqueous stabilization composition (Composition 43) provided effective stabilization for at least 5 days at ambient temperature.

### 5.6 Example 5

This Example demonstrates that an aqueous composition comprising PVP and an apoptosis inhibitor shows effective preservation of cfDNA without loss of yield compared to commercially-available blood preservation tubes.

The following commercially-available cell-free preservation blood tubes were tested: PAXgene Blood ccfDNA Tube (“Qiagen,” Cat. No. 768115, Qiagen), Cell-free DNA BCT (“St-DNA,” Cat. No. 218962, Streck), and Cell-Free RNA BCT (“St-RNA,” Cat. No. 218976, Streck). Vacutainer K₂EDTA Blood Collection Tube (“K₂EDTA,” Cat. No. 22-253-145, Becton-Dickinson) was used as non-stabilizing blood collection tube control condition.

9 ml of whole blood was added to an evacuated tube containing 1 ml of the aqueous composition (Composition 34). Blood was simultaneously added to Qiagen, St-DNA, St-RNA and K₂EDTA commercially-available blood collection tubes in the amounts indicated in the manufacturer protocols. The samples were prepared in duplicate and were incubated at ambient temperature (20-25° C.) for up to 7 days.

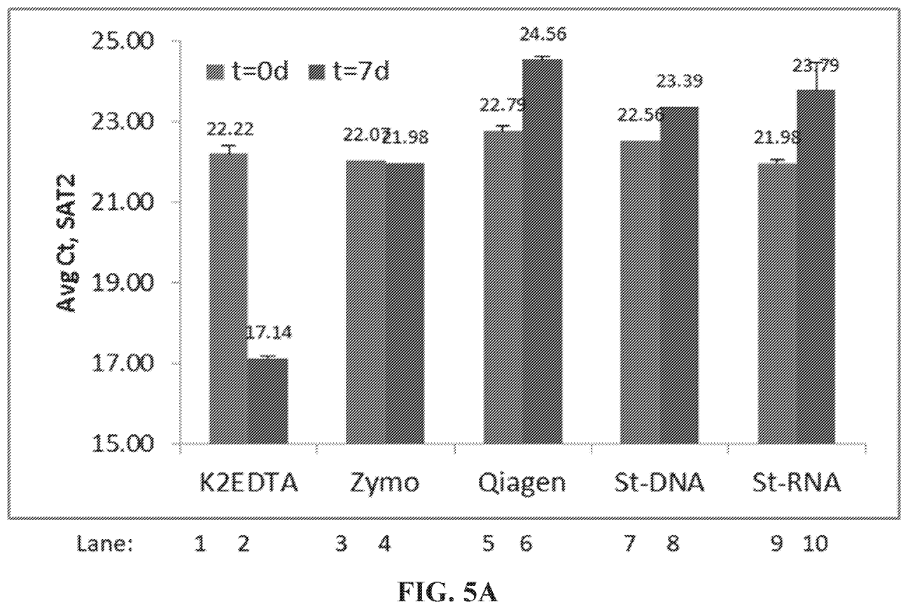

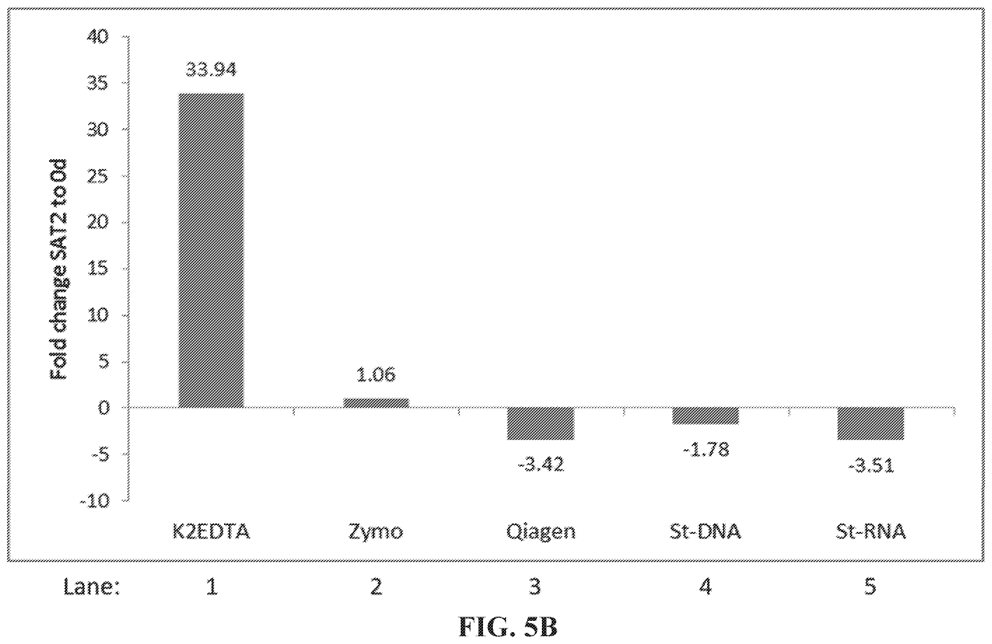

Samples were collected immediately (t=0 d) and after a 7-day incubation at ambient temperature (t=7 d) and plasma was separated described in Method 1. Plasma cfDNA was isolated as described in Method 3. Quantification of plasma cfDNA was performed using quantitative PCR targeting the Sat2 locus fragment as described in Method 5 (FIG. 5A). Cycle threshold (Ct) values obtained from quantitative PCR were also plotted as the fold change in Sat2 quantity detected after 7 days of incubation compared to Sat2 detected after 0 days (FIG. 5B).

### Quantitative PCR analysis of DNA

Blood collected into a K₂EDTA tube resulted in a Ct decrease over the course of the incubation at ambient temperature, from 22.22 at t=0 d to 17.14 at t=7 d (FIG. 5A, lanes 1 and 2, respectively). This decrease translates to a 33.94-fold increase in Sat2 locus fragment detected in the cfDNA (FIG. 5B, lane 1). The significant increase in Sat2 locus fragment observed at t=7 d indicates that the K₂EDTA tube provided little protection of cfDNA, highly diluting the originally present cfDNA with cellular DNA content spillage.

Blood collected into an evacuated blood tube containing 1 ml of the aqueous composition (Composition 43) maintained stable levels of Sat2 locus fragment. The Ct remained stable, with a value of 22.07 at t=0 d and 21.98 at t=7 d (FIG. 5A, lanes 3 and 4, respectively), which translates to a 1.06-fold increase in Sat2 detected (FIG. 5B, lane 2).

Blood preserved in tubes from Qiagen and Streck suffered from varying degrees of loss of Sat2 locus fragment signal after a 7 day incubation at ambient temperature (FIG. 5A, lanes 5-10). Incubation of blood in a PAXgene Blood ccfDNA Tube resulted in an increase of Ct from 22.79 to 24.56 (FIG. 5A, lanes 5 and 6, respectively), which translates to 3.42 fold decrease in Sat2 detected (FIG. 5B, lane 3; Qiagen). Incubation of blood in a Streck-DNA BCT tube resulted in an increase of Ct from 22.56 to 23.39 (FIG. 5A, lanes 7 and 8, respectively), which translates to 1.78 fold decrease (FIG. 5B, lane 4). Blood incubated in a Streck-RNA BCT tube resulted in an increase of Ct from 21.98 to 23.79 (FIG. 5A, lanes 9 and 10), which translates to 3.51 fold decrease in Sat2 fragment detected (FIG. 5B, lane 5). These results indicate that incubation of blood in these commercially-available blood preservation tubes resulted in reduced amounts of recovered plasma cfDNA after blood samples were incubated for 7 days at ambient temperature. The decrease observed in Qiagen and Streck blood tubes may be due to degradation over time of cfDNA present prior to the incubation.

Taken together, these results demonstrate that the aqueous composition comprising PVP and an apoptosis inhibitor (Composition 43) outperformed the preservative effect of other existing technologies by maintaining the yield of Sat2 present in cfDNA. In particular, the aqueous composition (Composition 43) shows effective preservation of cfDNA without loss of yield compared to tested commercially-available blood preservation tubes.

### 5.7 Example 6

This Example demonstrates that aqueous compositions comprising a combination of polymers, such as PVP-10000 and dmPEG-2000, and an apoptosis inhibitor can preserve cfRNA in blood.

Red blood cells make up approximately 50% of blood volume and, although they lack gDNA, contain high amounts of cellular RNA. Accordingly, the inhibition of red blood cell lysis (hemolysis) is critical to the preservation of cfRNA (e.g., by reducing contamination from cellular RNA). To determine if aqueous compositions comprising a combination of PVP and other types of polymers enhanced cfRNA stabilization, aqueous compositions comprising PVP as the sole polymer compared to compositions comprising PVP with other additional polymers were assessed for their ability to stabilize cfRNA.

Aqueous compositions containing the following polymer(s) were prepared: 15% w/v PVP-10000 (Composition 43); 30% w/v PVP-10000 (Composition 44); 15% w/v PVP-10000 and 15% w/v PEG-3350 (Composition 45); 15% w/v PVP-10000 and 15% w/v dmPEG-2000 (Composition 46); and 15% w/v PVP-10000 and 15% w/v PVP-40000 (Composition 47). The following components were present in all compositions: 137 mM NaCl; 2.7 mM KCl; 500 μM Q-VD-OPh; 100 mM K₂EDTA; 10 mM D+glucose; and 10 mM trehalose. Saline solution (Composition 1) (137 mM NaCl and 2.7 mM KCl) was used as non-stabilizing control.

The concentrations of components in each aqueous composition are listed in Table 7 below (middle column). Whole blood was mixed with the aqueous compositions at a ratio of 9 parts blood to 1 part aqueous composition to produce the resulting mixtures described in Table 7 (right column). The resulting mixtures were incubated at ambient temperature (20-25° C.) for up to 7 days.

TABLE 7
Aqueous compositions comprising PVP-10000, apoptosis inhibitor, anticoagulant,
and sugar in the presence or absence of an additional polymer

| | Concentration of | Final concentration of components |
|---|---|---|
| Aqueous | components in | (after addition of blood to |
| Composition | aqueous composition | composition in ratio of 9:1) |
| Composition 1 | 137 mM NaCl; 2.7 mM KCl | 13.7 mM NaCl; 0.27 mM KCl |
| (“saline solution”) | | (Saline) |
| Composition 43 | 137 mM NaCl; 2.7 mM KCl; | 13.7 mM NaCl; 0.27 mM KCl; |
| | 15% w/v PVP-10000; | 1.5% w/v PVP-10000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose | 1 mM trehalose (PVP-10k 1.5%) |
| Composition 44 | 137 mM NaCl; 2.7 mM KCl; | 13.7 mMNaCl; 0.27 mM KCl; |
| | 30% w/v PVP-10000; | 3% w/v PVP-10000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose | 1 mM trehalose (PVP-10k 3%) |
| Composition 45 | 137 mM NaCl; 2.7 mM KCl; | 13.7 mM NaCl; 0.27 mM KCl; |
| | 15% w/v PVP-10000; | 1.5% w/v PVP-10000; |
| | 15% w/v PEG-3350; | 1.5% w/v PEG-3350; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose | 1 mM trehalose (PVP-10k 1.5%, PEG-3350 1.5%) |
| Composition 46 | 137 mM NaCl; 2.7 mM KCl; | 13.7 mM NaCl; 0.27 mM KCl; |
| | 15% w/v PVP-10000; | 1.5% w/v PVP-10000; |
| | 15% w/v dmPEG-2000; | 1.5% w/v dmPEG-2000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose | 1 mM trehalose (PVP-10k 1.5%, dmPEG-2000 1.5%) |
| Composition 47 | 137 mM NaCl; 2.7 mM KCl; | 13.7 mM NaCl; 0.27 mM KCl; |
| | 15% w/v PVP-10000; | 1.5% w/v PVP-10000; |
| | 15% w/v PVP-40000; | 1.5% w/v PVP-40000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose | 1 mM trehalose (PVP-10k 1.5%, PVP-40k 1.5%) |

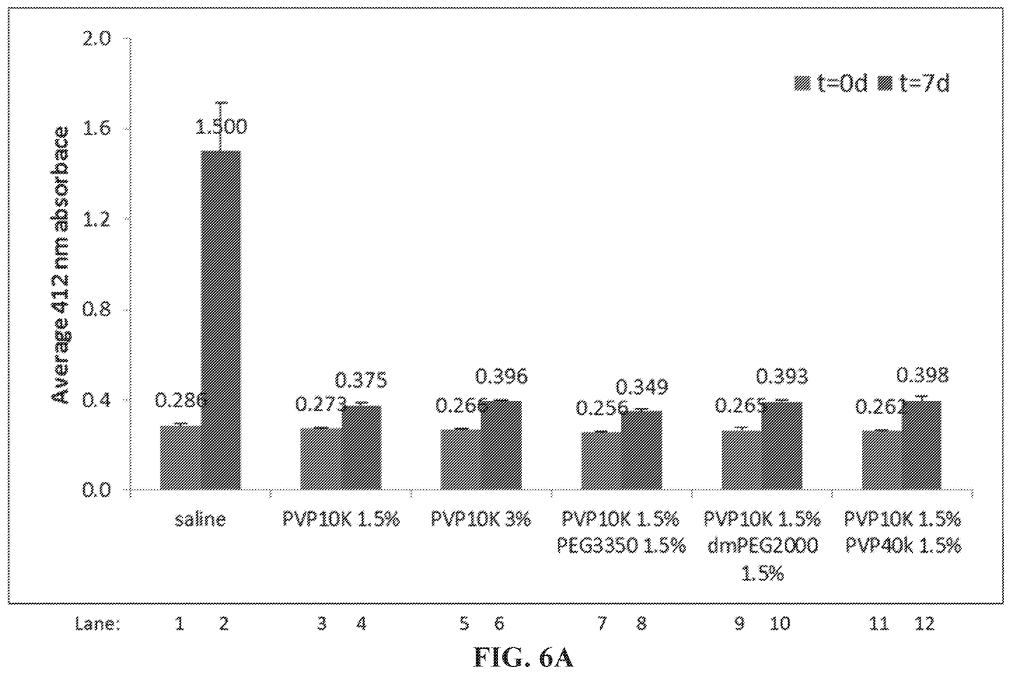

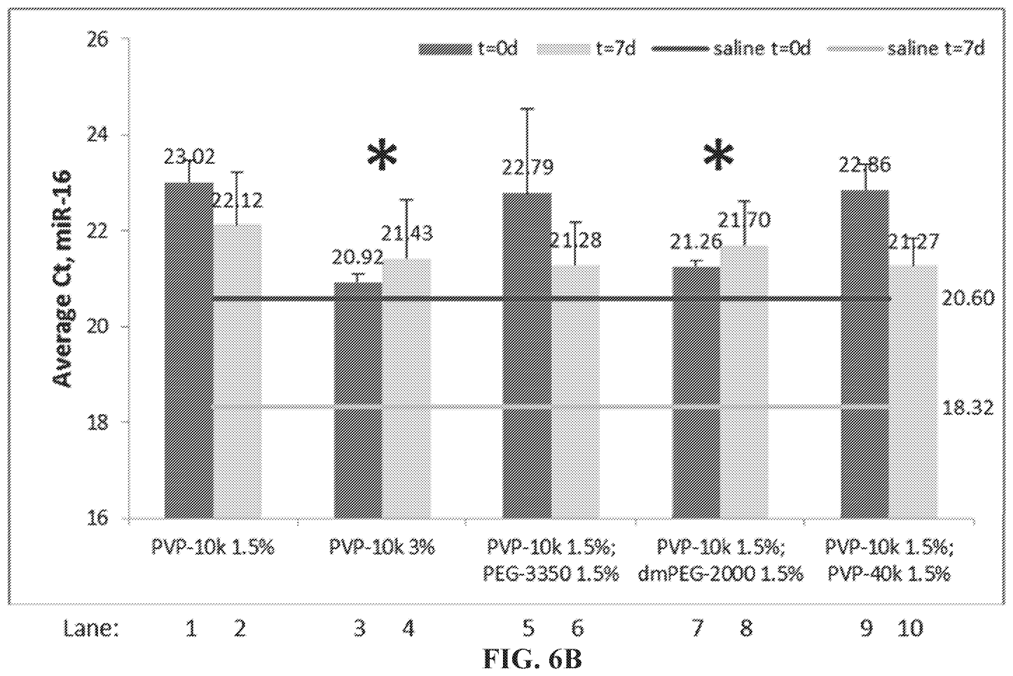

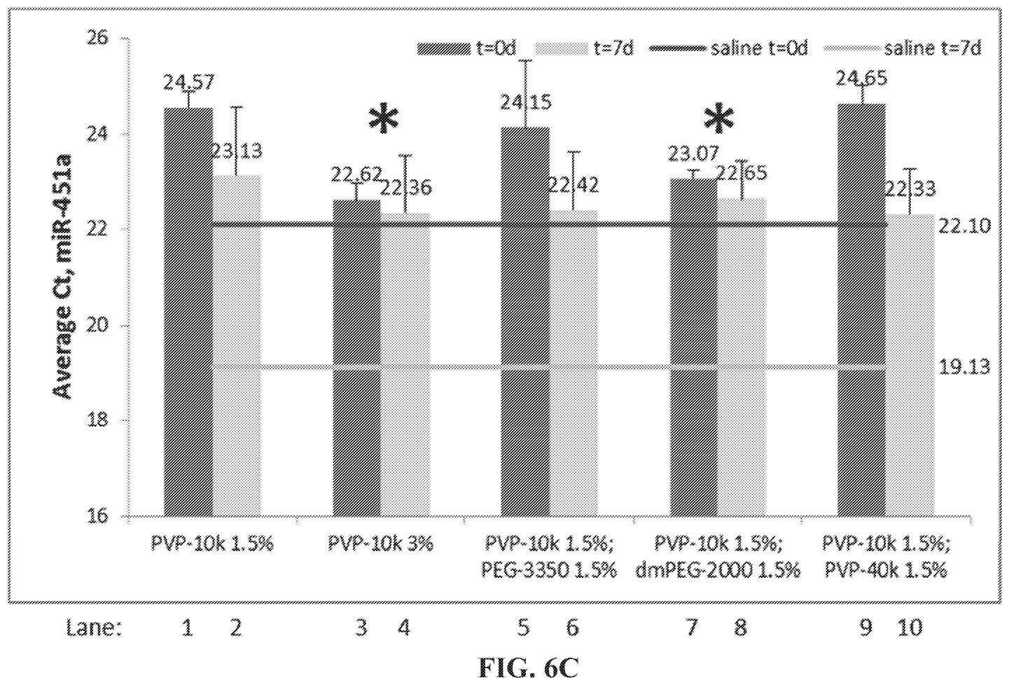

Plasma separation was performed as described in Method 1, and hemolysis was assessed by UV-Vis spectrophotometry as described in Method 2 (FIG. 6A). Plasma cfRNA was isolated as described in Method 6. cfRNA was quantified using reverse transcriptase quantitative PCR (rt-qPCR) targeting two microRNAs, hsa-miR-16-5p and hsa-miR-451a as described in Method 7 (FIGS. 6B and 6C). The microRNA hsa-miR-16-5p is highly abundant as a plasma cfRNA, and was examined to assess the stability of a cfRNA originally present in the plasma. The microRNA hsa-miR-451a is highly abundant in red blood cells, and its presence in cfRNA is indicative of its release due to hemolysis.

### Analysis of Blood Hemolysis in Plasma

The blood samples incubated with the aqueous compositions listed in Table 7 were assessed for hemolysis. When a blood sample was incubated with saline solution (Composition 1) for 7 days, the A₄₁₂ increased from 0.286 to 1.5, indicating an increase in hemolysis (FIG. 6A, lanes 1 and 2). In contrast, blood samples incubated with aqueous compositions comprising PVP-10000, or PVP-10000 with an additional polymer, such as PVP-40000 or dmPEG-2000, and an apoptosis inhibitor (Compositions 43-47), exhibited an inhibition of hemolysis (FIG. 6A, lanes 7-12).

### Analysis of Purified cfRNA

Purified cfRNA from plasma samples was assessed by reverse transcription quantitative PCR (rt-qPCR) of microRNAs hsa-miR-16-5p and hsa-miR-451 a. The average of duplicates for each sample was used to obtain the cycle threshold (Ct) value at t=0 d and t=7 d. Ct value indicates how many cycles are required to detect a rt-qPCR signal and is inversely correlated to the amount of target miRNA in the sample.

Ct values that show little change between t=0 d and t=7 d indicate that the miRNA was stable during incubation. rt-qPCR of hsa-miR-16-5p, a microRNA highly abundant in plasma, identified varying degrees of relative yield differences for blood incubated with the different compositions tested (FIGS. 6B and 6C). Blood incubated with saline solution (Composition 1) resulted in a decrease in the Ct for hsa-miR-16-5p from 20.60 at t=0 d to 18.32 at t=7 d, indicating an increase in the amount of microRNA (FIG. 6B; top and bottom horizontal lines, respectively).

Blood incubated with an aqueous composition comprising 15% w/v PVP-10000 and 15% w/v PVP-40000 (Composition 47) resulted in a decrease in Ct from 22.86 at t=0 d to 21.27 at t=7 d, indicating an increase in the amount of hsa-miR-16-5p detected (FIG. 6B; samples 11 and 12). However, when blood was incubated with an aqueous composition comprising 15% w/v PVP-10000 and 15% w/v dmPEG (Composition 46), the hsa-miR-16-5p Ct showed little change, increasing from 21.26 at t=0 d to 21.70 at t=7 d, suggesting stability of hsa-miR-16-5p (FIG. 6B, lanes 9 and 10).

The rt-qPCR of hsa-miR-451a, a microRNA highly enriched red blood cells, was assayed as an indicator of hemolysis. Blood incubated with a saline solution (Composition 1) resulted in a significant increase in the amount of hsa-miR-451a, as Ct values decreased from 22.10 at t=0 d to 19.13 at t=7 d, signifying release of the miRNA following hemolysis (FIG. 6C; top and bottom horizontal lines, respectively). However, when blood was incubated with an aqueous composition comprising 15% w/v PVP-10000 and 15% w/v dmPEG (Composition 46), the hsa-miR-451a Ct showed little change, decreasing from 23.07 at t=0 d to 22.65 at t=7 d (FIG. 6C, lanes 7 and 8).

The two aqueous compositions that resulted in the most effective stabilization of hsa-miR-16-5p and hsa-miR-451a in blood samples, as observed by rt-qPCR, are indicated by asterisks (FIGS. 6B and 6C). These data demonstrate that aqueous compositions comprising 30% w/v PVP-10000 (Composition 44) or 15% w/v PVP-10000 and 15% w/v dmPEG-2000 (Composition 47) can stabilize cfRNA in blood.

### 5.8 Example 7

This Example demonstrates that aqueous compositions comprising PVP, an apoptosis inhibitor, and one or more eryptosis inhibitors, such as caffeine and naringin, can significantly inhibit hemolysis.

Programmed cell death of red blood cells occurs through a cellular pathway called eryptosis and is distinct from apoptosis, which occurs in most other cell types including white blood cells. To determine if inhibition of eryptosis can enhance the stabilization of cfRNA, blood was incubated with aqueous compositions comprising exemplary eryptosis inhibitors.

Aqueous compositions comprising the following eryptosis inhibitors were prepared: 10 mM or 50 mM urea (Compositions 48 and 49, respectively); 40 μM or 200 μM naringin (Compositions 50 and 51, respectively); and 100 μM or 500 μM caffeine (Compositions 52 and 53, respectively). The following components were present in all compositions: 137 mM NaCl; 2.7 mM KCl; 15% w/v PVP-10000; 15% w/v dmPEG-2000; 500 μM Q-VD-OPh; 100 mM K₂EDTA; 10 mM D+glucose; and 10 mM trehalose (Composition 46). Saline solution (Composition 1) (137 mM NaCl and 2.7 mM KCl) was used as a non-stabilizing control.

The concentrations of components in each aqueous composition are listed in Table 8 (middle column). Whole blood was mixed with the aqueous compositions at a ratio of 9 parts blood to 1 part aqueous composition to produce the resulting mixtures described in Table 8 (right column). The resulting mixtures were incubated at ambient temperature (20-25° C.) for up to 5 days.

TABLE 8
Aqueous compositions comprising PVP-10000, apoptosis inhibitor, anticoagulant,
and sugar in the presence or absence of an additional polymer

| | Concentration of | Final concentration of components |
|---|---|---|
| Aqueous | components in | (after addition of blood to |
| Composition | aqueous composition | composition in ratio of 9:1) |
| Composition 1 | 137 mMNaCl; 2.7 mM KCl | 13.7 mM NaCl; 0.27 mM KCl |
| (“saline solution”) | | (Saline) |
| Composition 46 | 137 mM NaCl; 2.7 mM KCl; | 13.7 mM NaCl; 0.27 mM KCl; |
| | 15% w/v PVP-10000; | 1.5% w/v PVP-10000; |
| | 15% w/v dmPEG-2000; | 1.5% w/v dmPEG-2000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose | 1 mM trehalose |
| Composition 48 | 137 mM NaCl; 2.7 mM KCl; | 13.7 mM NaCl; 0.27 mM KCl; |
| | 15% w/v PVP-10000; | 1.5% w/v PVP-10000; |
| | 15% w/v dmPEG-2000; | 1.5% w/v dmPEG-2000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose; 10 mM urea | 1 mM trehalose; 1 mM urea |
| Composition 49 | 137 mM NaCl; 2.7 mM KCl; | 13.7 mMNaCl; 0.27 mMKCl; |
| | 15% w/v PVP-10000; | 1.5% w/v PVP-10000; |
| | 15% w/v dmPEG-2000; | 1.5% w/v dmPEG-2000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose; 50 mM urea | 1 mM trehalose; 5 mM urea |
| Composition 50 | 137 mM NaCl; 2.7 mM KCl; | 13.7 mM NaCl; 0.27 mM KCl; |
| | 15% w/v PVP-10000; | 1.5% w/v PVP-10000; |
| | 15% w/v dmPEG-2000; | 1.5% w/v dmPEG-2000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose; | 1 mM trehalose; |
| | 40 μM naringin | 4 μM naringin |
| Composition 51 | 137 mM NaCl; 2.7 mM KCl; | 13.7 mM NaCl; 0.27 mM KCl; |
| | 15% w/v PVP-10000; | 1.5% w/v PVP-10000; |
| | 15% w/v dmPEG-2000; | 1.5% w/v dmPEG-2000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose; | 1 mM trehalose; |
| | 200 μM naringin | 20 μM naringin |
| Composition 52 | 137 mM NaCl; 2.7 mM KCl; | 13.7 mM NaCl; 0.27 mM KCl; |
| | 15% w/v PVP-10000; | 1.5% w/v PVP-10000; |
| | 15% w/v dmPEG-2000; | 1.5% w/v dmPEG-2000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose; | 1 mM trehalose; |
| | 100 μM caffeine | 10 μM caffeine |
| Composition 53 | 137 mM NaCl; 2.7 mM KCl; | 13.7 mM NaCl; 0.27 mM KCl; |
| | 15% w/v PVP-10000; | 1.5% w/v PVP-10000; |
| | 15% w/v dmPEG-2000; | 1.5% w/v dmPEG-2000; |
| | 500 μM Q-VD-OPh; | 50 μM Q-VD-OPh; |
| | 100 mM K₂EDTA; | 10 mM K₂EDTA; |
| | 10 mM D + Glucose; | 1 mM D + Glucose; |
| | 10 mM trehalose; | 1 mM trehalose; |
| | 500 μM caffeine | 50 μM caffeine |

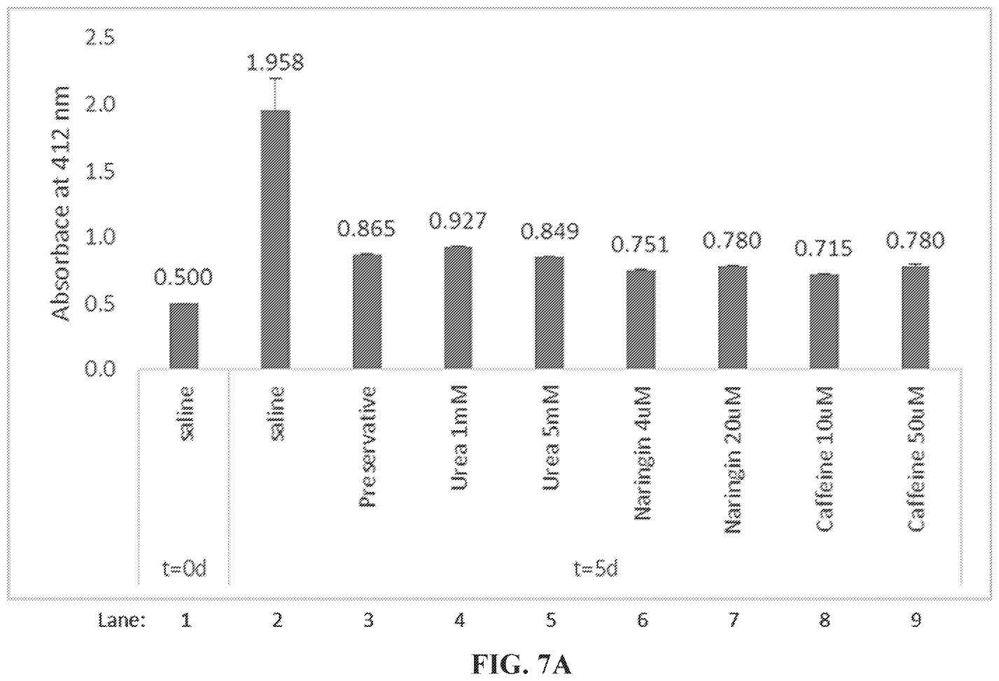

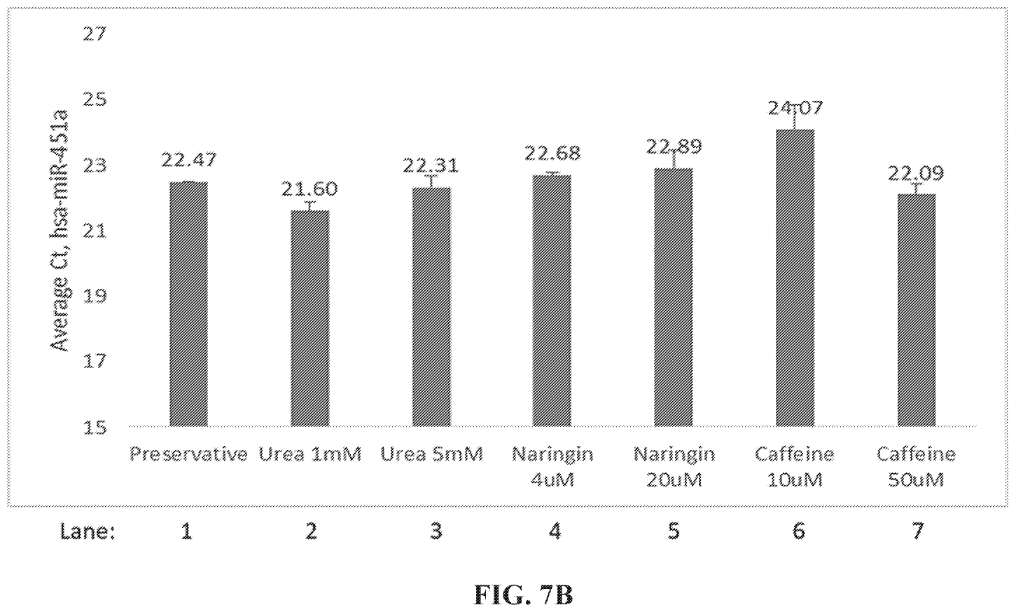

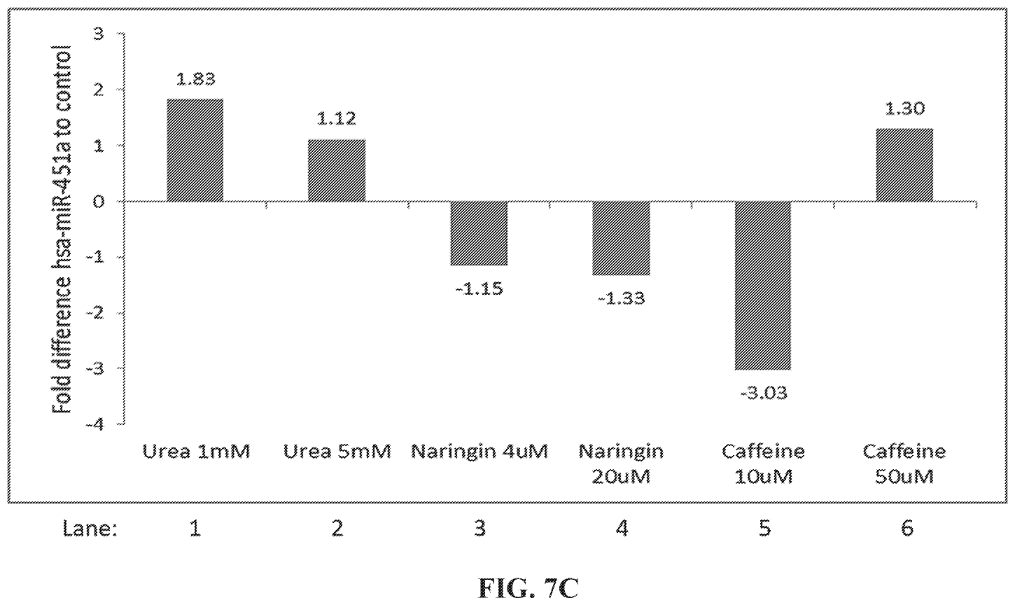

Blood samples were collected immediately (t=0 d) or after a 5-day incubation at ambient temperature (t=3 d) and plasma was separated as described in Method 1. Hemolysis assessment was done using UV-Vis spectrophotometry as described in Method 2 (FIG. 7A). Hemolysis assessment by rt-qPCR of hsa-miR-451a was done on cfRNA isolated from plasma samples as described in Method 7 (FIGS. 7B and 7C).

### Analysis of Blood Hemolysis in Plasma

The blood samples incubated with the aqueous compositions listed in Table 8 were assessed for hemolysis. Plasma absorbance was compared to plasma from blood incubated with saline solution, which exhibited an A₄₁₂ of 1.958 at t=7 (FIG. 7A, lane 2). Incubation of blood with an aqueous composition lacking eryptosis inhibitor (Composition 46), resulted in an A₄₁₂ of 0.865 (FIG. 7A, lane 3), showing inhibition of hemolysis. However, incubation of blood with all tested aqueous compositions comprising additional eryptosis inhibitors (Compositions 49-53) showed improvement in hemolysis inhibition compared to the aqueous composition lacking eryptosis inhibitor (Composition 46) (FIG. 7A, lane 3).

### Analysis of Purified cfRNA

Hemolysis was also assessed by quantifying the amount of hsa-miR-451a present in the plasma of blood samples incubated with aqueous compositions in the presence and absence of eryptosis inhibitors. The average of duplicates for each sample was used to obtain the cycle threshold (Ct) value (FIG. 7B). The fold-difference in the calculated amount of hsa-miR-451a in the aqueous composition containing eryptosis inhibitor (Compositions 48-53) and the aqueous composition lacking eryptosis inhibitor (Composition 46) was also determined (FIG. 7C), calculated by normalizing Ct values to the control, composition lacking eryptosis inhibitor, using 2\hat{ }(Normalization: experimental−control).

Incubation of blood with aqueous composition lacking eryptosis inhibitor (Composition 46) resulted in a Ct of 22.47 (FIG. 7B, lane 1). Incubation of blood with aqueous compositions comprising certain eryptosis inhibitor showed a decrease in the amount of hsa-miR-451a (e.g., an increased cycle threshold value) compared to aqueous composition (Composition 46). For example, an aqueous composition comprising 100 μM caffeine (Composition 52) reduced the amount of plasma hsa-miR-451a by 3.03-fold compared to aqueous composition lacking eryptosis inhibitor (Composition 46) (FIG. 7C, lane 5). These results show that addition of certain eryptosis inhibitors in aqueous composition (Composition 46) can decrease hemolysis, as assessed by absorbance and q-rtPCR of miRNA-451a.

Overall, these data demonstrate that incubation of blood at ambient temperature with aqueous compositions comprising eryptosis inhibitors such as caffeine (at 100 μM to 500 μM) and naringin (at 40 to 200 μM) can significantly inhibit hemolysis.

What is claimed is:

1. An aqueous composition for the stabilization of a cell-free nucleic acid (cfNA) population in a blood sample, the aqueous composition comprising a polyvinylpyrrolidone (PVP), an apoptosis inhibitor and one or more eryptosis inhibitors.

2. The aqueous composition of claim 1, wherein the PVP has a weight average molecular weight of from about 10,000 to about 40,000 Daltons, as determined by a light scattering technique.

3. The aqueous composition of claim 1, wherein the PVP is present in an amount of about 5% w/v to about 30% w/v of the aqueous composition.

4. The aqueous composition of claim 1, wherein the apoptosis inhibitor is a caspase inhibitor.

5. The aqueous composition of claim 1, wherein the apoptosis inhibitor is selected from the group consisting of quinolyl-valyl-O-methylaspartyl-[-2,6-difluorophenoxy]-methyl ketone (Q-VD-OPh), carbobenzoxy-valyl-alanyl-aspartyl-[O-methyl]-fluoromethylketone (Z-VAD-FMK) and Boc-Asp(OMe)-fluoromethyl ketone (BOC-D-FMK).

6. The aqueous composition of claim 1, wherein the apoptosis inhibitor is quinolyl-valyl-O-methylaspartyl-[-2,6-difluorophenoxy]-methyl ketone (Q-VD-OPh).

7. The aqueous composition of claim 5, wherein the Q-VD-OPh has a concentration of about 50 μM to about 5000 μM.

8. The aqueous composition of claim 1, wherein the one or more eryptosis inhibitors is selected from the group consisting of an inhibitor of increased intracellular Ca2+ activity, an inhibitor of ceramide formation, and an inhibitor of ATP depletion.

9. The aqueous composition of claim 1, wherein the one or more eryptosis inhibitors is selected from the group consisting of adenosine, amitriptyline, caffeine, a catecholamine, D4476, dibutyryl-cGMP, dithiothreitol, ethylisopropylamiloride, erythropoietin, flufenamic acid, furosemide, glutathione, 7-monohydroxyethylrutoside, N-acetylcysteine, naringin, 1,2,3,4-tetrahydro-6-nitro-2,3-dioxo-benzo[f]quinoxaline-7-sulfonamide, 6-cyano-7-nitroquinoxaline-2,3-dione (CNQX), niflumic acid, nitroprusside, 5-nitro-2-(3-phenylpropylamino)benzoic acid (NPPB), papanonoate, P38 Inh III, resveratrol, (R)-DRF503, salidroside, 4-(4-fluorophenyl)-2-(4-methylsulfinylphenyl)-5-(4-pyridyl)imidazole (SB203580), Staurosporine, Trolox, urea, vitamin E, xanthohumol, and Zidovudine.

10. The aqueous composition of claim 8, wherein one or more of urea, naringin and caffeine are present in the aqueous composition, and if present, the urea has a concentration of about 4 mM to about 650 mM, the naringin has a concentration of about 40 nM to about 40 μM, and the caffeine has a concentration of about 2 μM to about 500 μM.

11. The aqueous composition of claim 1, wherein the aqueous composition further comprises polyethylene glycol dimethyl ether (dmPEG).

12. The aqueous composition of claim 11, wherein the dmPEG has a weight average molecular weight of from about 250 to about 4000 Daltons, as determined by a light scattering technique.

13. The aqueous composition of claim 11, wherein the dmPEG is present in an amount about 5% w/v to about 30% w/v of the aqueous composition.

14. The aqueous composition of claim 1, wherein the aqueous composition is a saline composition.

15. The aqueous composition of claim 1, further comprising an anticoagulant.

16. The aqueous composition of claim 15, wherein the anticoagulant is a chelator.

17. The aqueous composition of claim 16, wherein the chelator is EDTA dipotassium salt (K₂EDTA).

18. The aqueous composition of claim 17, wherein the K₂EDTA has a concentration of about 10 mM to about 1000 mM.

19. The aqueous composition of claim 1, further comprising a sugar.

20. The aqueous composition of claim 19, wherein the sugar is selected from the group consisting of glucose, lactose, fructose, and galactose.

21. The aqueous composition of claim 19, wherein the sugar is glucose and the concentration of the glucose is about 10 mM and about 1000 mM.

22. The aqueous composition of claim 1, wherein the aqueous composition is used for research or diagnostic purposes.

23. A kit for stabilizing a cell-free nucleic acid (cfNA) population in a blood sample comprising the aqueous composition of claim 22, a blood collection container and instructional materials.


SEQUENCE LISTING (6 sequences)

SEQ ID NO: 1 - DNA, 24 bp, Artificial Sequence
  forward primer sequence for Sat2
catcaatcggatggaaacgaatgg

SEQ ID NO: 2 - DNA, 24 bp, Artificial Sequence
  reverse primer sequence for Sat2
ttcgcgaccattggatgattgcag

SEQ ID NO: 3 - DNA, 17 bp, Artificial Sequence
  forward primer sequence for hsa-miR-16-5p
cgcagtagcagcacgta

SEQ ID NO: 4 - DNA, 24 bp, Artificial Sequence
  reverse primer sequence for hsa-miR-16-5p
cagtttttttttttttttcgccaa

SEQ ID NO: 5 - DNA, 17 bp, Artificial Sequence
  forward primer sequence for hsa-miR-451a
cgcagaaaccgttacca

SEQ ID NO: 6 - DNA, 28 bp, Artificial Sequence
  reverse primer sequence for hsa-miR-451a
ttcagtttttttttttttttaactcagt